SEQUENCE LISTING (12 sequences)

SEQ ID NO: 1 - DNA, 873 bp, Ideonella sp.
  source 1..873: mol_type = genomic DNA
atgaactttccccgcgcttcccgcctgatgcaggccgccgttctcggcgggctgatggccgtgtcggccgccgccaccgcccagaccaacccctacgcccgcggcccgaacccgacagccgcctcactcgaagccagcgccggcccgttcaccgtgcgctcgttcaccgtgagccgcccgagcggctacggcgccggcaccgtgtactaccccaccaacgccggcggcaccgtgggcgccatcgccatcgtgccgggctacaccgcgcgccagtcgagcatcaaatggtggggcccgcgcctggcctcgcacggcttcgtggtcatcaccatcgacaccaactccacgctcgaccagccgtccagccgctcgtcgcagcagatggccgcgctgcgccaggtggcctcgctcaacggcaccagcagcagcccgatctacggcaaggtcgacaccgcccgcatgggcgtgatgggctggtcgatgggcggtggcggctcgctgatctcggcggccaacaacccgtcgctgaaagccgcggcgccgcaggccccgtgggacagctcgaccaacttctcgtcggtcaccgtgcccacgctgatcttcgcctgcgagaacgacagcatcgccccggtcaactcgtccgccctgccgatctacgacagcatgtcgcgcaatgcgaagcagttcctcgagatcaacggtggctcgcactcctgcgccaacagcggcaacagcaaccaggcgctgatcggcaagaagggcgtggcctggatgaagcgcttcatggacaacgacacgcgctactccaccttcgcctgcgagaacccgaacagcacccgcgtgtcggacttccgcaccgcgaactgcagctga

SEQ ID NO: 2 - DNA, 1647 bp, Ideonella sp.
  source 1..1647: mol_type = genomic DNA
atgatggcagcaaaagacgtggttttcggcggtgatgcgcgcgcgcgcatggtcgaaggcgtgaacatcctggccaacgccgtcaaggtcaccctcggcccgaagggccgcaacgtggtgctggagcgctcgttcggcgcccccaccgtcaccaaggacggtgtctcggtcgccaaggagatcgagctcaaggacaagctgcagaacatgggcgcccagatggtcaaggaagtcgcttccaagaccagcgacaacgccggtgacggcacgacgaccgccaccgtgctggcccaggccatcgtgcgcgaaggcatgaagtacgtggccgccggcatgaacccgatggacctgaagcgcggcatcgaccgtgccgtcgccgcgctggtcgagcagttgaagaaggcctcgaaggcgacgacgacctcgaaggaaatcgcccaggtcggcgcgatctcggccaacagcgacgagtcgatcggcaagatcatcgccgacgcgatggacaaggtcggcaaggaaggcgtcatcaccgtcgaggacggcaagtcgctggacaacgagctggacgtcgtcgaaggcatgcagttcgaccgcggctacctgtcgccctacttcatcaacaacccggacaagcaggctgccatcctggacaacccgttcgtcctgctgttcgacaagaagatcagcaacatccgcgacctgctgccgacgctggaagccgtcgccaaggccggccgtccgctgctgatcatcgccgaggaagtcgaaggcgaggcgctggcgaccctggtggtcaacaccatccgcggcatcctgaaggtcgccgccgtcaaggcgcccggcttcggtgaccgccgcaaggccatgctggaggacatcgcgatcctgaccggcggcaaggtgatcgccgaggaagtgggcctgtcgctcgagaaggtcacgctggccgacctgggccaggccaagcgcgtcgaggtggcgaaggaaaacaccacgatcatcgacggcgccggcgccgcggccgacatcgaggcccgcgtcaagcagatccgtgtccagatcgaggaagccaccagcgactacgaccgcgagaagctgcaggagcgtgtcgcgaagctggccggcggcgtggccgtcatccgcgtcggtgccgccaccgaagtcgagatgaaggagaagaaggcccgcgtcgaagacgcgctgcacgccacgcgtgccgccgtcgaggaaggcatcgtcgccggtggtggcgtcgcgctgctgcgtgcgaagcagtcggcgggcgagatcaagggcgacaacgccgaccaggacgccggcatcaagctggtgctgaaggcgatcgaggccccgctgcgcgagatcgtctacaacgccggtggcgaagcctccgtcgtcgtgaacgccgtgctggccggcaagggcaactacggcttcaacgccgccaacgacacctatggcgacatgatcgagatgggcatcctggacccgaccaaggtgacccgcaccgcgctgcagaacgccgcgtcggtggcctcgctgatgctgaccaccgagtgcatggtcgccgaggcgccgaaggacgaggcgcccgccccggccggcggcatgggcggcatgggtggcatgggcggcatggacatgtga

SEQ ID NO: 3 - DNA, 621 bp, Ideonella sp.
  source 1..621: mol_type = genomic DNA
atgctgcgtggacctgccaatgaacccactggcgcctctgcggcggcagccagtagctcggtcatggtggccgatgcttcgggcgcaaacctggacgcggcgcgcttatttgagttgggattcgcgggtgggctggtgattgatcgcgatacacgggcagtcattgaggcagtattaaattcgatgccggaacagccctcggctgatgacctgcaacgactggagcgtacacttcgtgaaggactgcctcgtgaagatgcggagcgagctttaaaattatttggaagctatcgcgattatactgcggacgtgcggcgacaaatggaacccctgggggttccgcgtaatctgcaagaaatgaatgcattctttgatcagatggaggcgattaaacaacgtcatttcgacgcggcgaccgcgcaagcgctgtttggaccggcggatatgcatgctcgtgtgagtatggaagccatgtttgttgatcaggatccatctttaaccttagagcagaaaaaacagcggctcgatgagctcagagcaaagctgccgccggatcaacgtagcctgatccctgaaccatcccagccggcctcactcgagcaccaccaccaccaccactga

SEQ ID NO: 4 - DNA, 1647 bp, Escherichia coli
  source 1..1647: mol_type = genomic DNA
atggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgggtggcatgggcggcatgatgtaa

SEQ ID NO: 5 - DNA, 645 bp, Rhizobacter sp.
  source 1..645: mol_type = genomic DNA
atgcaagcggcggcgaaagcgggtcggtcaccagcttccggtgctggcggggcagccccttgggtggtgaccgttagtgatgcaatgggcgcaactatggatgcatctcgcttatttgaattgggctttgcgggcgggcttgtcattgataaggacactcgtgcatcagtcgaagcattgctgaatagtatgccggaggatctttctgaaacagatctggctcgtctggagcgtaccttacgtgaaggtctgccaaaggaagatgccgaaaaagcgtacaagttaatcactgactaccgcagttacacaaaggacattcgcgacgagatgcaaccaaaaggcatcccaggtaatatgcaggaggcgcgcacttttttcgaccagatggaagcagtaaagcgtcgtcacttcgacgaagccacggcaaacgctttgtttgggcaagctgacttttacgcacgtttgtcgatggaagctatgttcgtgcaacaagacactacattgaccgatgaacaaaaaaaggcgcaacttgatgctttgcgtgctcagctgcccgcagatcagaaaagtttaattccacaggacggtcccccaccggcggcatcgcaacccgcatcactcgagcaccaccaccaccaccactaa

SEQ ID NO: 6 - DNA, 594 bp, Rhizobacter sp.
  source 1..594: mol_type = genomic DNA
atgagccaacccgcttcgacaggtgatagcaagttagttgaaattgatttgcttgggacaaaactggacgccgcccgcttgttcgacttaggcttcgctggtggattgaacattgatcaacatacccgctcgacgttggacaccctgctgatgaacatgtctgacacacccgcggcacaagagatcgagaaattggaatggaccttgcgtaacggattgccgaaggatgacgccgagaaggcaattaaaatgtttcacggctatcgtgcatacctgggtgacatgaaaggtgagttgcagcgcatgggtattcccgagactccagcggcagctaacgcgtacttcgatcaactggcgcttatgcagcgccgccatttcgatgacacaaccgcagcggctttgtttgggcaggaaaatcagaacgcacgtcttgtcatgcaagcagctcttatcacccaaaatgaagcgttatcagccagcgagaagaaggaacaacttgacttattacgcacccagttgcccgagggtaaacgtgatcttatcccagcgaccgaaccagccaaacctctcgagcaccaccaccaccaccactaa

SEQ ID NO: 7 - DNA, 1032 bp, unidentified
  source 1..1032: mol_type = genomic DNA
atgtcgaaatacataatattacttgttctggctgtggccctttcacttacaattatgcaaagtcgtagcactccaggtcccgctgcggtagcagagtcgccgtcagcgacttcccctgcaacaacagcccccacaccaccccgtcaacctttgaaagcgcagccggaaccccaaggaaccccgcagttaccgaccagcttcaaaggaacccaggtagatggacaattccgcctggacgaagccggcaaccttttaattggagcggaattacgccatctgtttgattacttcttagcagcagcaggagaagaacctcttaaaaactcgattgagcgtcttcgtcgttatattgtcgctgagttaccagaaccggctcaaagtcaagcctcggctgtattaacacagtaccttaattataaacgccagcttttggatgtagaagcaacttattcacgcatcaccgatatttcagctttgcgccagcgcctgagcgccgtacaggcgttacgcgcgcgcgtgttagaacccgcagtccaccaggctttctttgctttggacgaagcctatgatcgctttagcctggaacgcttagctattcaagcagactcagcattagatagcgaagcaaagggtcgtgcgatcgaccagctgcgcgccggattaccaggggaccttcaggaacttctggttccacaattacagtcggagttgcgtgagcaaacagttgctttgcaggcccaaggtgcaaatgctcagcaaatccgccagctgcgccaacagttggtggggtcagaagccgccacccgcttggaggcattggaccgtcagcgtgaacaatggcagcagcgcgttgcagtttatcgtcaagaacgtcagcgtattgaagcaacgcgcggattggacgacgtggagcgccgctctgctatcgaacgccttgaggcggagcaattcgacgagggagaacgtctgcgccttgtcgcagcgtttcagcaacaggaagttgcagagcgccaccaccaccaccaccactaa

SEQ ID NO: 8 - DNA, 1083 bp, Pseudomonas sp.
  source 1..1083: mol_type = genomic DNA
atgagtaaattctttagtctttcattggtcgcagtagtggtagctggaggattgactctgtattggcgctggcccgcggcagtccccgaggtgctgcccaccgctgcggttgcagtgcccagtcgtcagacgtccactgagcccctttcagttcctatcgccaaagcctcatccgaggctcaaccgtcagtagcagaaagccttccctctctggtggacacagaagttgatgggcagttacgtacagacgcagccgggaatttagtgttagacttagcagttcgtgattattttgactactttcttagcgcggtggatcactccggccttgatgcagtaatcgaagcgttacttgccgacgctggacgtcgccttcccgagcctgctcttggccaaatgatctctttgcttggagattatctggactataaacgcgccagtatggctttgatgcagcagccattggacgcgcaacagcaggtcgaacccaaagcacaattacaggctttgcagtctgcatttgctcgcctggacgaattgcgccgtgcccatttctccgcagccgcgcaagaggcgttgttcggagcagagcaggcatacgctcgctacaccttagattctcttgctttacaacaacgtgacgatcttggcgaggcgcaacgtgcgcagcgtctggaacagcttcgcgaacgtcttcccgaggcattgcgtgaatctgagcagcgtcaacaactggcgttggagcaacttcaacgtagcgagcaactttggcgtgatggcgctgacgagcaacaagtacgtgagttcttggcgatgacctatgaccctcagactgtacagcgtttactggacgagcaacgtcgcgaacgtgattggcagcaacattatcaggcttaccgcaacgagctggcttctttgcaagggcgcggtttatctgaagctgatggtgagcagcttcagcgtcaacttcgcgaacgccttttttcgtcagaagatcgtcatcgtgtggaaacgtacgatgctatcgctgcaaaacagccagaaccattggaccatctcgagcaccaccaccaccaccactaa

SEQ ID NO: 9 - DNA, 801 bp, synthetic construct
  source 1..801: mol_type = other DNA
atgcagaccaatccgtatgcgcgcggtccgaatccgacagccgccagtttggaagcgagcgctggtccattcaccgttcgctcctttaccgtgagtagaccgagcggttatggcgctggcaccgtttactatccaacaaatgctgggggtaccgtgggcgccatagccatagttcccgggtatacggcacggcagtcatcaattaaatggtggggaccgcgtctggcatcccacggtttcgtagtaattacaattgacacaaattccacgtttgactatccatcaagtcggagttcgcaacaaatggccgcgctgcgccaggtggcgtcgttaaacggtgacagtagcagcccgatttacggaaaggtcgataccgctcgtatgggtgttatggggcatagtatgggaggtggagcatccctgcgatctgctgctaacaacccttcgctgaaagcagcgattcctcaagcaccatgggattctcaaacaaattttagttctgtaactgtgcccacgctgatcttcgcatgtgaaaacgatagtatagccccggtcaactctcatgcacttcctatctatgattctatgtcacgcaacgctaagcagtttctcgaaattaatggtggctcacattcctgtgcgaatagcggcaattctaaccaagcattaatcggaaaaaaaggcgttgcatggatgaaacgttttatggacaatgatactaggtattctacttttgcctgcgagaacccgaatagcaccgcagtgtctgattttcgtacagcgaattgcagcctcgagtga

SEQ ID NO: 10 - DNA, 801 bp, synthetic construct
  source 1..801: mol_type = other DNA
atgcagaccaatccgtatgcgcgcggtccgaatccgacagccgccagtttggaagcgagcgctggtccattcaccgttcgctcctttaccgtgagtagaccgagcggttatggcgctggcaccgtttactatccaacaaatgctgggggtaccgtgggcgccatagccatagttcccgggtatacggcacggcagtcatcaattaaatggtggggaccgcgtctggcatcccacggtttcgtagtaattacaattgacacaaattccacgttagaccagccagaaagtcggagttcgcaacaaatggccgcgctgcgccaggtggcgtcgttaaacggtacaagtagcagcccgatttacggaaaggtcgataccgctcgtatgggtgttatggggtggagtatgggaggtggaggctccctgatctctgctgctaacaacccttcgctgaaagcagcggcgcctcaagcaccatggcactcttcgacaaattttagttctgtaactgtgcccacgctgatcttcgcatgtgaaaacgatagtatagccccggtcaactcttcagcacttcctatctatgattctatgtcacgcaacgctaagcagtttctcgaaattaatggtggctcacattcctgtgcgaatagcggcaattctaaccaagcattaatcggaaaaaaaggcgttgcatggatgaaacgttttatggacaatgatactaggtattctacttttgcctgcgagaacccgaatagcaccgcagtgtctgattttcgtacagcgaattgcagcctcgagtga

SEQ ID NO: 11 - DNA, 1224 bp, Aquabacterium parvum
  source 1..1224: mol_type = genomic DNA
atgggcagcagccatcatcatcatcatcacagcagcggcctggtgccgcgcggcagccatatggctagcatgactggtggacagcaaatgggtcgcggatccgaattcaagcttatgaggagcgatgccttctacacgccgccccagccgttgccgagcgggccccatggcacgctgattcgctcccgtccattcattccggttggattgctggcttacggctggcaggtcatgtacaagtccaacgacgtcaatggcttgccgattgccatcaccggcaccgtgctggtgccctttacgccatggctctccggcccccggccggttattgcgtgggctccaggaacgcagggccaggccgacaactgcgcgccctcgcaccagtacgcgctgagcacggaatatgaagcgacgattgccggcgcgacgccggcaaccctcgcccgcggttgggcgatcgcgatgacggactatcagggtcttggtacaccgggcgatcacacctgggggatcggcaaggccgaagggcaagccgtcctcgacgcggcactggcggcgcagcagttgagcggggcgggcctgtcggcgaacgcccccgtcggcatcatgggctattcccagggtggccatgcagcggctttcgcggccgaactgcagcccgcatatgcccccggtctgaatgtgcggggcgttgccgcaggagctgggccattcagcatcgctgatgtatatgcgaacgttaatggtggcccgttcggcggggtagggccgattgcgctgatgggcctgaatgccgcctaccctgaacttggcctctcatcgatcctcacaccctatgggcagtcggtgattgccgactttcgcggcacaaaatgcctgcttgacgtgactgccagctatccgttcctgtctgactcggtcctcgtccaggctccagccatcctgaaccgcgccgactggctggcgcgcatggcgcagaaccagagcggcaatggcgtgcctgccgtaccggcattggtgtacgcgggaaccctcgacgagatcatcccctttgggcagaaccagaagctgtttgccgcctggtgtgcgcgcggtggcaacgtggcgttccggaccatcggcgcgaccgaacacgccactgggctgcttctcggcttgccggttgcactggactggatgacacagcgattcggcagtgtccctgcggtatccgagtgcccatag

SEQ ID NO: 12 - DNA, 840 bp, Ralstonia sp.
  source 1..840: mol_type = genomic DNA
atgaaaaatgctccgatccgttcagttttggctacttgtttggttgctgctgctgcttctgctttggctcaaactactgctccaacttctgcttctttgaatgcttctgctggtccattgtctgtttctactgcttctgttactgctccaagaggttttggtggtggtactatttattatccaactactcaagctagatatggtgttattgctatttctccaggttttactgctactcaaatttctattgcttggttgggtagaagaattgctactcatggttttgttgttattactttgaatactttgactactttggatcaaccagattctagagctaatcaattgatggctgctttgaatcatgttattaattcttcttcttctgctgttaaagctagaattgatacttctagattggctgttgctggtcattctatgggtggtggtggtgctttgattgctgctagagataatccaactttgaaagcttcttatccattgactccatggaatattaattctgcttttactactgttagagttccaactatgattgttggtgctgatggtgatactattgctccagttggtactcatgctagaccattttatgctgctttgccaggtgctacttctaaagcttatggtgaattgaatggtgctactcatttttctccaaattctactaatactccaattggtagatatggtgttgcttggatgaaaagatttttggatggtgatactagatattctacttttttgtgtggtactgaacatactagatatgctactgctctggtgttcgaccgctataatcagaactgcccatattga

(19) United States
(12) Patent Application Publication
(10) Pub. No.: US 2025/0145940 A1
(43) Pub. Date: May 8, 2025

(54) TRANSFORMED STRAIN AND METHOD FOR DEGRADING PET BY USING THE SAME

(71) Applicant: National Cheng Kung University, Tainan (TW)

(72) Inventors: I-Son NG, Tainan (TW); Jie-Yao YU, Tainan (TW); Wan-Wen TING, Tainan (TW); Chuan-Chieh HSIANG, Tainan (TW)

(21) Appl. No.: 18/938,396

(22) Filed: Nov. 6, 2024

Related U.S. Application Data
(60) Provisional application No. 63/547,394, filed on Nov. 6, 2023.

(51) Int. Cl. C12N 1/20; C08J 11/10; C12N 9/18; C12N 15/70; C12R 1/19

(52) U.S. Cl. CPC C12N 1/20; C08J 11/105; C12N 9/18; C12N 15/70; C12R 2001/19

(57) ABSTRACT

The present invention provides a transformed strain, comprising: a host cell; a first nucleotide sequence, located inside the host cell, the first nucleotide sequence encoding a PETase, which is derived from Ideonella; and a second nucleotide sequence, located inside the host cell, the second nucleotide sequence comprising a first chaperon nucleotide sequence or a second chaperon nucleotide sequence; wherein the first chaperon nucleotide sequence encodes a molecular chaperon protein (GroELS), and the second chaperon nucleotide sequence encodes a lipase secretion chaperone protein (LsC). The present invention also provides a method for degrading plastic, using said transformed strain for degrading a plastic having PET.

### CROSS-REFERENCE TO RELATED APPLICATION

This application claims the benefit of provisional application Ser. No. 63/547,394, filed Nov. 6, 2023. The disclosure of the above application is incorporated herein in its entirety by reference.

The sequence information contained in the Sequence Listing XML file, with the file name “PI-112-159-US-Sequence-20241226.xml” created on Dec. 26, 2024 and having a file size of 23,059 bytes, is incorporated herein in its entirety by reference.

### FIELD OF THE INVENTION

The present invention relates to the field of biotechnology and material recycling technology, especially relates to transformed strain and method for degrading PET by using the same.

### BACKGROUND OF THE INVENTION

Modern technology and life, including food industry, manufacture industry and medical industry, heavily rely on plastics. Polyethylene terephthalate (PET), having excellent physical properties, durability, and portability, is one of plastics that are widely used. That is to say, the use of the plastics containing PET provides convenience in modern life, but it also leads to problems related to the waste plastics.

Further, under the premise that modern people cannot avoid using plastics, the waste plastic problem has become an important issue. The traditional methods for addressing the waste plastic problem comprises landfill and incineration. The landfill, is not the good solution for sure, the soil practically degrades most of the plastic at a very slow rate, the landfill actually occupies large amount of land resources, and the waste plastics may release hazardous substances into the soil during the degradation. On the other hand, although the incineration can rapidly eliminate the volume of the waste plastics, the waste plastics still has potential to release hazardous substances into the air if the incineration is conducted under impropriate conditions, further, both the landfill and the incineration have no opportunity to reuse the waste plastics, therefore both of them are not the proper choice for the modern society with rising awareness of sustainability.

Accordingly, there should be a technical solution for reusing the waste plastics. In recent, enzymatic degradation has emerged as a better choice for taking cake of the waste plastics, based on the specific structure of the enzyme, the enzymatic degradation usually doesn't require high temperature for degrading the waste plastic, thereby reduce the fuel cost associated with the high temperature process; Moreover, the plastic monomer generated from the waste plastics through enzymatic degradation, can be further used to produce a new plastic, in other words, the enzymatic degradation gives the waste plastics market competitiveness, and therefore has the potential for development.

However, the cost of the enzymatic degradation remains high, the reason comprising the solubility, activity, and stability of the enzyme remains unideal, which make it hard to demonstrate the advantage of the enzymatic degradation for degrading waste plastics, and need to be improved.

### SUMMARY OF THE INVENTION

In order to solve the problem mentioned above, the present invention provides a transformed strain, comprising: a host cell; a first nucleotide sequence located inside the host cells, the first nucleotide sequence encoding a PETase originated from Ideonella, and a second nucleotide sequence located inside the host cell, the second nucleotide comprising a first chaperone nucleotide sequence or a second chaperone nucleotide sequence, wherein the first chaperone nucleotide sequence encodes a molecular chaperone protein (GroELS), the second chaperone nucleotide sequence encodes a lipase secretion chaperone protein (LsC), and at least one of the first nucleotide sequence and the second nucleotide sequence is exogenous.

In some embodiments, the host cell is Escherichia coli.

In some embodiments, the PETase further originated from Escherichia, Pichia, Bacillus, Pseudomonas, Stutzerimonas, Streptomyces, Aquabacterium, Ralstonia, or Rhizobacter.

In some embodiments, the molecular chaperone protein is originated from Escherichia, Ideonella, Pichia, Bacillus, Pseudomonas, Stutzerimonas, Streptomyces, Aquabacterium, Ralstonia, or Rhizobacter.

In some embodiments, the lipase secretion chaperone protein is originated from Escherichia, Ideonella, Pichia, Bacillus, Pseudomonas, Stutzerimonas, Streptomyces, Aquabacterium, Ralstonia, or Rhizobacter.

In some embodiments, the first nucleotide sequence comprises: a nucleotide sequence having at least 70% sequence identity to SEQ ID NO.: 1 and same activity with the PETase, a nucleotide sequence having at least 70% sequence identity to SEQ ID NO.: 9 and same activity with the PETase, or a nucleotide sequence having at least 70% sequence identity to SEQ ID NO.: 10 and same activity with the PETase.

In some embodiments, the transformed strain further comprising a first vector and a second vector, the first vector and the second vector are located inside the host cell respectively, wherein the first nucleotide sequence is integrated at the first vector, the second nucleotide sequence is integrated at the second vector, and the first vector is different from the second vector.

In some embodiments, the first vector has a first promoter, the second vector has a second promoter, the first promoter is identical to the second promoter.

In some embodiments, the first vector is pET28a, the second vector is pSB4A3.

In some embodiments, the first promoter is T7 promoter, the second promoter is T7 promoter.

In some embodiments, the first chaperone nucleotide sequence comprises: a nucleotide sequence having at least 70% sequence identity to SEQ ID NO.: 2 and same activity with the molecular chaperone protein, or a nucleotide sequence having at least 70% sequence identity to SEQ ID NO.: 4 and same activity with the molecular chaperone protein.

In some embodiments, the second chaperone nucleotide sequence comprises: a nucleotide sequence having at least 70% sequence identity to SEQ ID NO.: 3 and same activity with the lipase secretion chaperone protein, a nucleotide sequence having at least 70% sequence identity to SEQ ID NO.: 5 and same activity with the lipase secretion chaperone protein, a nucleotide sequence having at least 70% sequence identity to SEQ ID NO.: 6 and same activity with the lipase secretion chaperone protein, a nucleotide sequence having at least 70% sequence identity to SEQ ID NO.: 7 and same activity with the lipase secretion chaperone protein, a nucleotide sequence having at least 70% sequence identity to SEQ ID NO.: 8 and same activity with the lipase secretion chaperone protein.

In some embodiments, the transformed strain further comprising a third vector, located inside the host cell, used for increasing a tRNA expression level of the host cell.

In some embodiments, the third vector is pRARE.

In some embodiments, the second chaperone nucleotide sequence comprises: a nucleotide sequence having at least 70% sequence identity to SEQ ID NO.: 3 and same activity with the lipase secretion chaperone protein, a nucleotide sequence having at least 70% sequence identity to SEQ ID NO.: 5 and same activity with the lipase secretion chaperone protein, a nucleotide sequence having at least 70% sequence identity to SEQ ID NO.: 6 and same activity with the lipase secretion chaperone protein, a nucleotide sequence having at least 70% sequence identity to SEQ ID NO.: 7 and same activity with the lipase secretion chaperone protein, a nucleotide sequence having at least 70% sequence identity to SEQ ID NO.: 8 and same activity with the lipase secretion chaperone protein.

The present invention further provides a method of preparing PETase, comprising: establishing a transformed strain as mentioned above; and expressing the transformed strain for obtaining the PETase.

The present invention further provides a method for degrading plastic, using the transformed strain as mentioned above for degrading a plastic, wherein the plastic has PET.

In some embodiments, the method for degrading plastic further comprising: an offering step: allowing the transformed strain to contact with a matrix, and inducing expression of the transformed strain so as to produce a PETase inside the transformed strain.

In some embodiments, the matrix comprises: solid media or liquid media.

In some embodiments, the method for degrading plastic further comprising: a releasing step: disrupting the transformed strain so as to release the PETase from the transformed strain and allow the PETase to contact with the matrix, and defining a degrading composition comprising the PETase and the matrix; and a degrading step: allowing the degrading composition to contact with the plastic.

### BRIEF DESCRIPTION OF THE DRAWINGS

FIG. 1A is a flowchart of transformed strain preparation.

FIG. 1B is a flowchart of PETase preparation.

FIG. 1C is a flowchart of plastic degradation method.

FIG. 2 is a plasmid map of pET28A-duet.

FIG. 3A is a plasmid map of pSB4A3-T7B-sfGFP.

FIG. 3B is a plasmid map of pSB4A3-T7OS-sfGFP.

FIG. 3C is a plasmid map of pSB4A3-lacI-T7OS-sfGFP.

FIG. 4A is a first result diagram of WT-PETase and molecular chaperone protein co-expression, wherein the molecular chaperone protein is EcGroELS. In X-axis, mono-expression group: Ø represents that only the WT-PETase is expressed, and the molecular chaperone protein is not expressed; co-expression group: “T7B” represents the plasmid “pSB4A3-T7B-sfGFP”, “T7OS” represents the plasmid “pSB4A3-T7OS-sfGFP”, “IT7OS” represents the plasmid “pSB4A3-lacI-T7OS-sfGFP”. At the upper part of the diagram, the protein expression level can be observed. The Y-axis represents the relative concentration of TPA (%).

FIG. 4B is a second result diagram of WT-PETase and molecular chaperone protein co-expression, wherein the molecular chaperone protein is IsGroELS. In X-axis, mono-expression group: Ø represents that only the WT-PETase is expressed, and the molecular chaperone protein is not expressed; co-expression group: “T7B” represents the plasmid “pSB4A3-T7B-sfGFP”, “T7OS” represents the plasmid “pSB4A3-T7OS-sfGFP”, “IT7OS” represents the plasmid “pSB4A3-lacI-T7OS-sfGFP”. At the upper part of the diagram, the protein expression level can be observed. The Y-axis represents the relative concentration of TPA (%).

FIG. 5A is a first result diagram of WT-PETase and lipase secretion chaperone protein co-expression, wherein the lipase secretion chaperone protein is IsLsC. In X-axis, mono-expression group: Ø represents that only the WT-PETase is expressed, and the lipase secretion chaperone protein is not expressed; co-expression group: “T7B” represents the plasmid “pSB4A3-T7B-sfGFP”, “T7OS” represents the plasmid “pSB4A3-T7OS-sfGFP”, “IT7OS” represents the plasmid “pSB4A3-lacI-T7OS-sfGFP”. At the upper part of the diagram, the protein expression level can be observed. The Y-axis represents the relative concentration of TPA (%).

FIG. 5B is a degradation product comparison diagram, X-axis represents mono-expression group and co-expression group respectively.

FIG. 5C is a first Scanning Electron Microscope (SEM) diagram, which represents the PET membrane degradation degree caused by mono-expression group.

FIG. 5D is a second Scanning Electron Microscope (SEM) diagram, which represents the PET membrane degradation degree caused by co-expression group.

FIG. 6A is an analysis result diagram of Sequence Similarity Network, (SSN).

FIG. 6B is a second result diagram of WT-PETase and lipase secretion chaperone protein co-expression. In X-axis, mono-expression group: Ø represents that only the WT-PETase is expressed, and the lipase secretion chaperone protein is not expressed; co-expression group: the lipase secretion chaperone protein comprises IsLsC, RsLsC1, RsLsC2, SsLsC, and PsLsC as shown in FIG. 6A The right part of the diagram represents a total concentration of (TPA+MHET) (mM), which is corresponding to the amount of degradation product represented in the main part of the diagram. The protein expression level, shown in the upper part of the diagram corresponding to the main part.

FIG. 6C is a third result diagram of WT-PETase and lipase secretion chaperone protein co-expression. In X-axis, mono-expression group: Ø represents that only the WT-PETase is expressed, and the lipase secretion chaperone protein is not expressed; co-expression group: the lipase secretion chaperone protein comprises IsLsC, RsLsC1, RsLsC2, SsLsC, and PsLsC as shown in FIG. 6A The right part of the diagram represents a total concentration of (TPA+MHET) (mM), which is corresponding to the amount of degradation product represented in the main part of the diagram. The protein expression level, corresponding to the main part and shown in the upper part of the diagram.

FIG. 7A is an expression level result diagram of PETase and lipase secretion chaperone protein co-expression, wherein the PETase comprises WT-PETase, Dura-PETase, and Thermo-PETase, the lipase secretion chaperone protein is IsLsC.

FIG. 7B is an activity level result diagram of PETase and lipase secretion chaperone protein co-expression, wherein the PETase comprises WT-PETase, Dura-PETase, and Thermo-PETase, the lipase secretion chaperone protein is IsLsC. In X-axis, IsLsC (−) represents mono-expression group, which expresses PETase only; IsLsC (+) represents co-expression group. The Y-axis represents the concentration of TPA (mM).

FIG. 8A is an expression level result diagram of lipase and lipase secretion chaperone protein co-expression, wherein the lipase comprises Ap-lipase, and Re-lipase; the lipase secretion chaperone protein is IsLsC.

FIG. 8B is an activity level result diagram of lipase and lipase secretion chaperone protein co-expression. In X-axis, IsLsC (−) represents mono-expression group, which expresses lipase only; IsLsC (+) represents co-expression group. The Y-axis represents the relative activity (%) to p-NPB.

### DETAILED DESCRIPTION OF THE INVENTION

The technical features of the present invention will be explained below through diagrams and embodiments.

In some embodiments, the present invention provides a “transformed strain”, the transformed strain expresses a “degradative enzyme” and a “chaperone protein”, wherein the degradative enzyme has the ability to degrade a “plastic”, the chaperone protein has the ability to: assist the degradative enzyme to achieve a folding state for the best catalyzing function, or assist the degradative enzyme to achieve a folding state for the most stable structure, so as to maintain or elaborate the activity or the expressing level of the degradative enzyme synthesized from the transformed strain.

In some embodiments, the present invention describes a method for preparing transformed strain, and describes a method for degrading the plastic by using the transformed strain, or by using the interior substances from the transformed strain, or by using the degradative enzyme.

As provided by the present invention, the “transformed strain” is produced by “introducing” a “vector” into a “host cell”, in other words, the vector located inside the transformed strain after the introduction. It is clear that, the “introduce” may refers to an electrical method, a physical method, a chemical method, or any combination thereof was performed, to increase the permeability of the host cell's cell membrane, or to increase the permeability of the host cell's cell wall, for sending the exogenous vector into the host cell. In general, the methods in use include those that are typically known to persons having ordinary skill in the art.

In some embodiments, the vector carries genes, the vector comprises: plasmid, viral vector, cosmid, or artificial chromosome, wherein the viral vector comprises phage, the artificial chromosome comprises bacterial artificial chromosome (BAC), yeast artificial chromosome (YAC), human artificial chromosome (HAC).

As previously mentioned, the reason that a transformed strain can express the degradative enzyme and the chaperone protein comprises: the transformed strain has a degradative enzyme gene and a chaperone enzyme gene. In detail, the degradative enzyme gene could be endogenous or exogenous, wherein the endogenous degradative enzyme gene may locate at the chromosome or in the plasmid of the host inherently, the exogenous degradative enzyme gene may locate at the vector. On the other hand, the chaperone protein gene could be endogenous or exogenous, wherein the endogenous chaperone protein gene may locate at the chromosome or in the plasmid of the host inherently, the exogenous chaperone protein gene may locate at the vector.

In some embodiments, the vector having a vector gene comprises: main functional gene, resistance gene, reporter gene, marker gene, promoter, origin of replication, ribosome binding site, restriction enzyme cutting site, regulon or any combination thereof, wherein the regulon comprises enhancer, silencer or any combination thereof. In some embodiments, the main functional gene transcripts a functional RNA or further translates a functional protein. In some embodiments, the main functional gene comprises the degradative enzyme gene or the chaperone protein gene. On one hand, those vector genes could be optionally and operatically combined or connected with each other, for example, there may be a number of gap sequence between the regulon and the promoter.

In some embodiments, the host cell is optional, the host cell could be a prokaryotic cell or a eukaryotic cell. In some embodiments, the prokaryotic cell originates from Escherichia, Ideonella, Pichia, Bacillus, Pseudomonas, Stutzerimonas, Streptomyces, Aquabacterium, Ralstonia, or Rhizobacter. The host cell could be pretreated or preestablished, so as to allow the vector to be introduced the host cell easily, or allow the vector to be maintained inside the host cell easily; in other words, the host cell could be a competent cell.

In some embodiments, the host cell contains N of vector A, wherein the N refers to a positive integer; In some embodiments, the host cell contains types of vectors, concretely, the host cell contains N of vector A, N of vector B, and N of vector C, and so on, but not limited to this, obviously, N refers to a positive integer, and A, B, C refers to different vector respectively.

In some embodiments, the degradative enzyme gene originates from Escherichia, Ideonella, Pichia, Bacillus, Pseudomonas, Stutzerimonas, Streptomyces, Aquabacterium, Ralstonia, or Rhizobacter.

In some embodiments, the chaperone protein gene originates from Escherichia, Ideonella, Pichia, Bacillus, Pseudomonas, Stutzerimonas, Streptomyces, Aquabacterium, Ralstonia, or Rhizobacter.

As previously mentioned, the “plastic” refers to those containing a plastic component; In some embodiments, the degradative enzyme has the ability to degrade the plastic or the plastic component, wherein the plastic component comprises polyethylene terephthalate (PET), high density polyethylene (HDPE), low density polyethylene (LDPE), polypropylene (PP), polyvinyl chloride (PVC), or polystyrene (PS).

In some embodiments, the degradative enzyme is a PETase, wherein the PETase can degrade the PET, thereby being referred to as an esterase; on the other hand, when the PETase degrades the PET, water molecules are involved, therefore the PETase is also referred to as a hydrolase.

Further, PET could be synthesized by terephthalic acid (TPA) and ethylene glycol (EG) through a condensation reaction, and PET could be degraded into Bis(2-Hydroxyethyl) terephthalate (BHET) or monohydroxyethyl terephthalate (MHET). Therefore, when the PETase is applied to PET, the activity of PETase can be measured by detecting TPA, EG, BHET, or MHET.

In some embodiments, the degradative enzyme is a lipase, the lipase could be referred to as an esterase, or a hydrolase.

As provided by the present invention, “sequence identity” is shown as a percentage, which represents a comparison result generated by comparing a standard nucleotide sequence with a candidate nucleotide sequence. In some embodiments, the sequence identity, under conditions that allow an introduction of gap sequences, is calculated by aligning the standard nucleotide sequence with the candidate nucleotide sequence. In fact, the Needleman-Wunsch Algorithm could be applied to calculate the sequence identity. In other hand, any of two codons may have codon degeneracy, for example, the codon that encodes a valine comprises “GTC” and “GTA”, wherein the C and the A should be considered different when calculating the sequence identity.

Understandably, multiple codons may indicate the same amino acid. Therefore, in some embodiments, the standard nucleotide sequence, and the candidate nucleotide modified based on the standard nucleotide sequence, may encode the same protein, for example the PETase or the chaperone protein as provided by the present invention. In other words, there are possibilities to have a protein having the same or almost the same activity of the PETase or the chaperone protein by changing the sequence in a rational range.

According to some embodiments, the present invention provides a transformed strain, comprising: a host cell; a first nucleotide sequence located inside the host cells, the first nucleotide sequence encoding a PETase originated from Ideonella, and a second nucleotide sequence located inside the host cell, the second nucleotide comprising a first chaperone nucleotide sequence or a second chaperone nucleotide sequence, wherein the first chaperone nucleotide sequence encodes a molecular chaperone protein (GroELS), the second chaperone nucleotide sequence encodes a lipase secretion chaperone protein (LsC), at least one of the first nucleotide sequence and the second nucleotide sequence is exogenous.

In some embodiments, the PETase comprises: WT-PETase, Dura-PETase, or Thermo-PETase.

In some embodiments, the host cell is Escherichia coli (E. coli) comprising: BL21(DE3), Rosetta(DE3), C41(DE3), C43(DE3), BL21-AI, Origami(DE3), or Lemo21(DE3).

In some embodiments, the first nucleotide sequence comprises: a nucleotide sequence having at least 70% sequence identity to SEQ ID NO.: 1 and same activity with the PETase, a nucleotide sequence having at least 70% sequence identity to SEQ ID NO.: 9 and same activity with the PETase, or a nucleotide sequence having at least 70% sequence identity to SEQ ID NO.: 10 and same activity with the PETase.

In some embodiments, the first nucleotide sequence is the nucleotide sequence as shown in SEQ ID NO.: 1, or is the nucleotide sequence at least having 70%, 75%, 80%, 85%, 90%, 95%, 98%, 99%, or 99.5% sequence identity and having the same activity to the nucleotide sequence as shown in SEQ ID NO.: 1, able to encodes the PETase: WT-PETase.

In some embodiments, the first nucleotide sequence is the nucleotide sequence as shown in SEQ ID NO.: 9, or is the nucleotide sequence at least having 70%, 75%, 80%, 85%, 90%, 95%, 98%, 99%, or 99.5% sequence identity and having the same activity to the nucleotide sequence as shown in SEQ ID NO.: 9, able to encodes the PETase: Dura-PETase.

In some embodiments, the first nucleotide sequence is the nucleotide sequence as shown in SEQ ID NO.: 10, or is the nucleotide sequence at least having 70%, 75%, 80%, 85%, 90%, 95%, 98%, 99%, or 99.5% sequence identity and having the same activity to the nucleotide sequence as shown in SEQ ID NO.: 10, able to encodes the PETase: Thermo-PETase.

In some embodiments, the transformed strain further comprising a first vector and a second vector, the first vector and the second vector are located inside the host cell respectively, wherein the first nucleotide sequence is integrated at the first vector, the second nucleotide sequence is integrated at the second vector, and the first vector is different from the second vector.

In some embodiments, the first vector has a first promoter, the second vector has a second promoter, the first promoter is identical to the second promoter.

In some embodiments, the first vector is pET28a, the second vector is pSB4A3.

In some embodiments, the first promoter is T7 promoter, the second promoter is T7 promoter.

In some embodiments, the first chaperone nucleotide sequence comprises: a nucleotide sequence having at least 70% sequence identity to SEQ ID NO.: 2 and same activity with the molecular chaperone protein, or a nucleotide sequence having at least 70% sequence identity to SEQ ID NO.: 4 and same activity with the molecular chaperone protein.

In some embodiments, the first chaperone nucleotide sequence is the nucleotide sequence as shown in SEQ ID NO.: 2, or is the nucleotide sequence at least having 70%, 75%, 80%, 85%, 90%, 95%, 98%, 99%, or 99.5% sequence identity and having the same activity to the nucleotide sequence as shown in SEQ ID NO.: 2, able to encodes the molecular chaperone protein: IsGroELS.

In some embodiments, the first chaperone nucleotide sequence is the nucleotide sequence as shown in SEQ ID NO.: 4, or is the nucleotide sequence at least having 70%, 75%, 80%, 85%, 90%, 95%, 98%, 99%, or 99.5% sequence identity and having the same activity to the nucleotide sequence as shown in SEQ ID NO.: 4, able to encodes the molecular chaperone protein: EcGroELS.

In some embodiments, the transformed strain further comprises a third vector, located inside the host cell, used for increasing a tRNA expression level of the host cell.

In some embodiments, the third vector is different from the first vector, the third vector is different from the second vector.

In some embodiments, the third vector is pRARE, wherein the pRARE is used for supplementing the tRNA inside the transformed strain.

In some embodiments, the second chaperone nucleotide sequence comprises: a nucleotide sequence having at least 70% sequence identity to SEQ ID NO.: 3 and same activity with the lipase secretion chaperone protein, a nucleotide sequence having at least 70% sequence identity to SEQ ID NO.: 5 and same activity with the lipase secretion chaperone protein, a nucleotide sequence having at least 70% sequence identity to SEQ ID NO.: 6 and same activity with the lipase secretion chaperone protein, a nucleotide sequence having at least 70% sequence identity to SEQ ID NO.: 7 and same activity with the lipase secretion chaperone protein, a nucleotide sequence having at least 70% sequence identity to SEQ ID NO.: 8 and same activity with the lipase secretion chaperone protein.

In some embodiments, the second chaperone nucleotide sequence is the nucleotide sequence as shown in SEQ ID NO.: 3, or is the nucleotide sequence at least having 70%, 75%, 80%, 85%, 90%, 95%, 98%, 99%, or 99.5% sequence identity and having the same activity to the nucleotide sequence as shown in SEQ ID NO.: 3, able to encodes the lipase secretion chaperone protein: IsLsC.

In some embodiments, the second chaperone nucleotide sequence is the nucleotide sequence as shown in SEQ ID NO.: 5, or is the nucleotide sequence at least having 70%, 75%, 80%, 85%, 90%, 95%, 98%, 99%, or 99.5% sequence identity and having the same activity to the nucleotide sequence as shown in SEQ ID NO.: 5, able to encodes the lipase secretion chaperone protein: RsLsC1.

In some embodiments, the second chaperone nucleotide sequence is the nucleotide sequence as shown in SEQ ID NO.: 6, or is the nucleotide sequence at least having 70%, 75%, 80%, 85%, 90%, 95%, 98%, 99%, or 99.5% sequence identity and having the same activity to the nucleotide sequence as shown in SEQ ID NO.: 6, able to encodes the lipase secretion chaperone protein: RsLsC2.

In some embodiments, the second chaperone nucleotide sequence is the nucleotide sequence as shown in SEQ ID NO.: 7, or is the nucleotide sequence at least having 70%, 75%, 80%, 85%, 90%, 95%, 98%, 99%, or 99.5% sequence identity and having the same activity to the nucleotide sequence as shown in SEQ ID NO.: 7, able to encodes the lipase secretion chaperone protein: SsLsC.

In some embodiments, the second chaperone nucleotide sequence is the nucleotide sequence as shown in SEQ ID NO.: 8, or is the nucleotide sequence at least having 70%, 75%, 80%, 85%, 90%, 95%, 98%, 99%, or 99.5% sequence identity and having the same activity to the nucleotide sequence as shown in SEQ ID NO.: 8, able to encodes the lipase secretion chaperone protein: PsLsC.

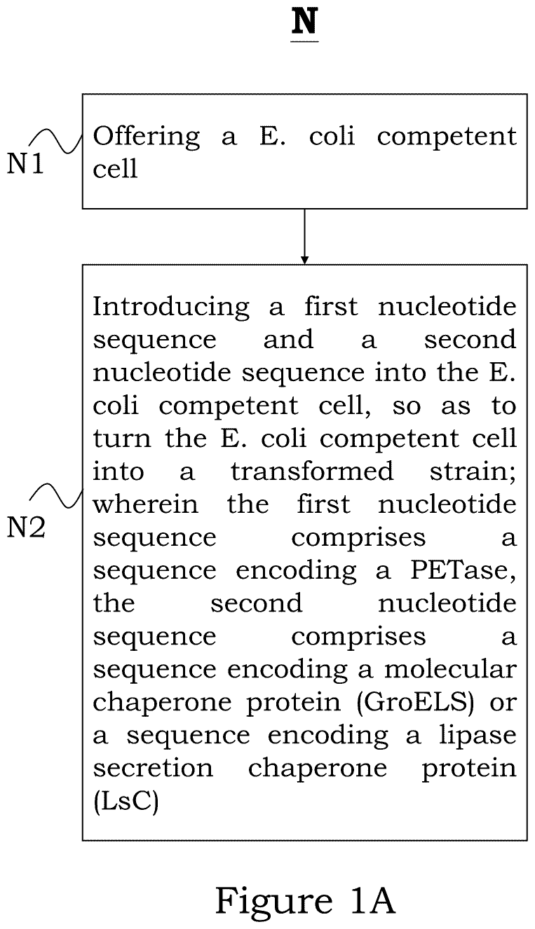

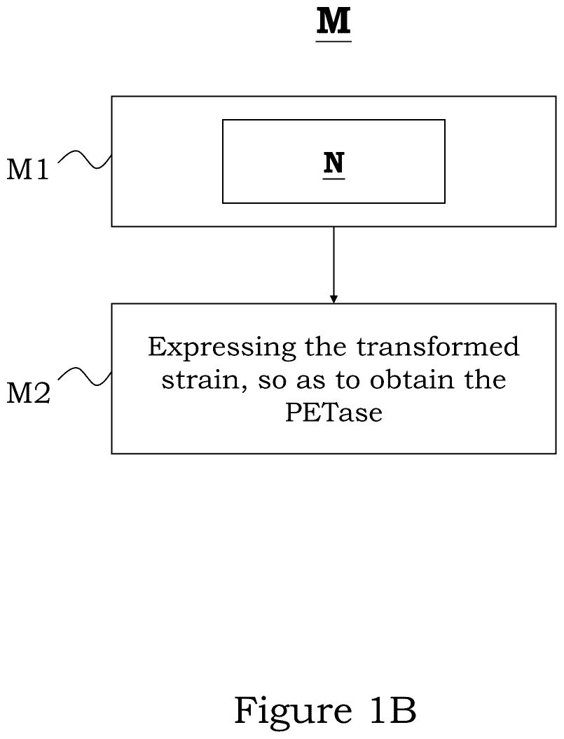

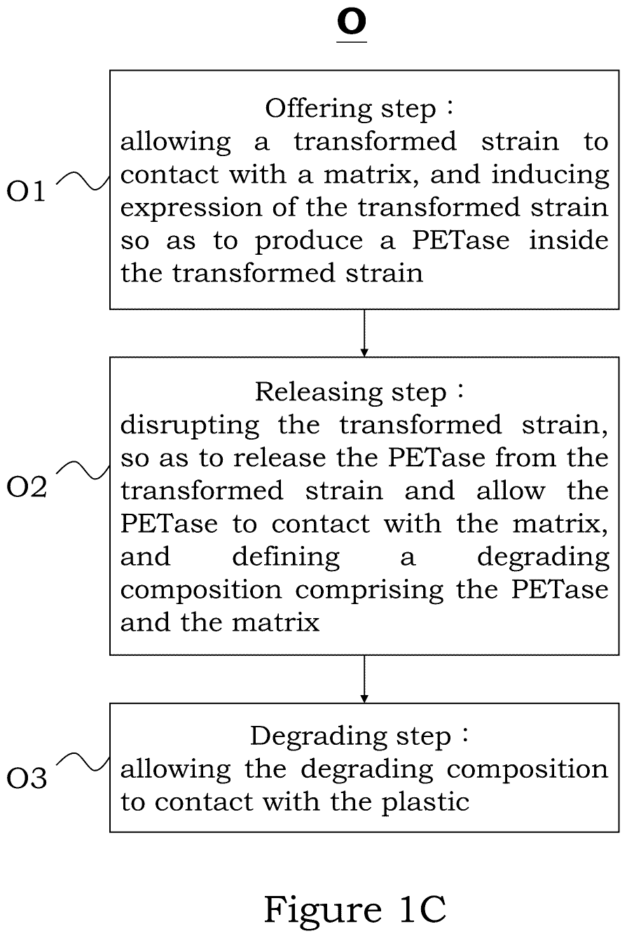

As shown in FIG. 1A, according to some embodiments, the present invention provides a method for preparing transformed strain (N), comprising: (N1): offering a E. coli competent cell; and (N2): introducing a first nucleotide sequence and a second nucleotide sequence into the E. coli competent cell, so as to turn the E. coli competent cell into a transformed strain, wherein the first nucleotide sequence comprises a sequence encoding a PETase, the second nucleotide sequence comprises a sequence encoding a molecular chaperone protein (GroELS) or a sequence encoding a lipase secretion chaperone protein (LsC).

As shown in FIG. 1B, according to some embodiments, the present invention provides a method for preparing PETase (M), comprising: (M1): using the method for preparing the transformed strain (N) for establishing the transformed strain; and (M2): expressing the transformed strain, so as to obtain the PETase. Concretely, in the (M2), it is allowed to use an inducer for expressing the transformed strain.

As shown in FIG. 1C, according to some embodiments, the present invention provides a method for degrading plastic (O), which comprising using the transformed strain to degrade a plastic, wherein the plastic has PET.

As shown in FIG. 1C, in some embodiments, comprising: an offering step (O1): allowing a transformed strain to contact with a matrix, and inducing expression of the transformed strain so as to produce a PETase inside the transformed strain, wherein the matrix comprises: solid media or liquid media; a releasing step (O2): disrupting the transformed strain, so as to release the PETase from the transformed strain and allow the PETase to contact with the matrix, and defining a degrading composition comprising the PETase and the matrix; and a degrading step (O3): allowing the degrading composition to contact with the plastic.

In some embodiments, the releasing step (O2) further comprising: disrupting the transformed strain by using a chemical method or by using a physical method, so as to release a transformed strain interior substance from the transformed strain, and allow the transformed strain interior substance to contact with the matrix for forming a degrading composition; and allowing the degrading composition to contact with the plastic, so as to degrade the plastic having PET. It is understood that, for releasing the interior substance from the transformed strain, and for keeping the activity or solubility of the PETase in the transformed strain interior substance, the chemical method comprises lysing the transformed strain by using a chemical reagent but not limited to this, or the physical method comprises breaking the transformed strain by using an ultrasonic or a blender but not limited to this.

Moreover, the transformed strain interior substance comprises the PETase. The matrix could be liquid form or solid form for accommodating, culturing, or maintaining the transform strain, also, the matrix could be liquid form or solid form for keeping the activity of the transformed strain interior substance, therefore the type or formation of the matrix is not limited, for example, the matrix comprises culture medium, water, or buffer such as phosphate buffered saline (PBS). In fact, the form of the substrate or the plastic containing PET, which can be degraded by PETase, is not limited; therefore, the plastic comprises sheet plastic component, powder plastic component, or solution containing plastic component.

In some embodiments, the method for degrading the plastic (O) further comprises a purifying step, which located between the releasing step (O2) and the degrading step (O3). With the purifying step, the PETase, the chaperone protein, or the lipase secretion chaperone protein can be separated from the transformed strain interior substance; and then those components could be applied to the plastic, so as to degrade the plastic component of the plastic. In general, the purifying step comprises using a column for purifying the PETase, the chaperone protein, or the lipase secretion chaperone protein; the type of column can be chosen according to the properties including size, electricity, or molecular marker (for example, His-tag of the PETase, the chaperone protein, or the lipase secretion chaperone protein). Furthermore, it should be considered that the 5′ end or 3′ end of the first nucleotide sequence or the second nucleotide sequence can be added with a tag sequence for the subsequent purifying step if needed.

In some embodiments, the method for degrading the plastic (O) further comprises: expressing the transformed strain; and applying the transformed strain to a plastic. In other words, the transformed strain can degrade the plastic without breaking the cell membrane or cell wall of the transformed strain, the PETase is secreted from the interior of the transform strain to the exterior of the transform strain.

According to some embodiments, the present invention provides a transformed strain, comprising: a host cell; a first nucleotide sequence located inside the host cells, the first nucleotide sequence encoding a lipase; and a second nucleotide sequence located inside the host cell, the second nucleotide comprising a first chaperone nucleotide sequence or a second chaperone nucleotide sequence, wherein the first chaperone nucleotide sequence encodes a molecular chaperone protein (GroELS), the second chaperone nucleotide sequence encodes a lipase secretion chaperone protein (LsC), and at least one of the first nucleotide sequence and the second nucleotide sequence is exogenous.

In some embodiments, the lipase comprises: Ap-lipase, or Re-Lipase.

In some embodiments, the first nucleotide sequence is the nucleotide sequence as shown in SEQ ID NO.: 11, or is the nucleotide sequence at least having 70%, 75%, 80%, 85%, 90%, 95%, 98%, 99%, or 99.5% sequence identity and having the same activity to the nucleotide sequence as shown in SEQ ID NO.: 11, able to encodes the lipase: Ap-lipase.

In some embodiments, the first nucleotide sequence is the nucleotide sequence as shown in SEQ ID NO.: 12, or is the nucleotide sequence at least having 70%, 75%, 80%, 85%, 90%, 95%, 98%, 99%, or 99.5% sequence identity and having the same activity to the nucleotide sequence as shown in SEQ ID NO.: 12, able to encodes the lipase: Re-lipase.

The technical features and functional features of the present invention will be further explained below through specific embodiments.

In the specific embodiments, the test of “protein expression level” is performed through western blot, which is well-known to the public.

In the specific embodiments, the “culture condition” refers to: using LB medium for culturing at 37° C. until the OD₆₀₀ reaches 0.6, wherein the culture is performed with shaking rate 200 rpm.

In the specific embodiments, the “whole transformed strain interior substance” is obtained by breaking the transformed strain in the LB medium, wherein the break is performed by using a high-pressure cell disruptor, the breaking condition is 30 kPsi.

In the specific embodiments, the “soluble protein from whole transformed strain interior substance” is obtained from the supernatant generated by centrifuging the whole transformed strain interior substance, wherein the centrifuge is performed at 12,000 rpm for 10 minutes.

In the specific embodiments, the “induction condition” comprises: the induction temperature is 22° C., the induction time is 24 hours, the induction is performed with shaking rate 200 rpm.

In the specific embodiments, the “test condition” comprises: using glycine-NaOH buffer (pH 9.0), the test degree is 50° C., the test time is 24 hours, the test is performed with shaking for testing the activity of the PETase.

In the specific embodiments, the “test condition” comprises: using Na₂HPO₄—HCl buffer (pH 7.0), the test degree is 25° C., the test time is 10 minutes, the test is performed with shaking for testing the activity of the lipase.

In the specific embodiments, the sequence shown as SEQ ID NO.: 1 encodes WT-PETase, SEQ ID NO.: 2 encodes IsGroELS, SEQ ID NO.: 3 encodes IsLsC, SEQ ID NO.: 4 encodes EcGroELS, SEQ ID NO.: 5 encodes RsLsC1, SEQ ID NO.: 6 encodes RsLsC2, SEQ ID NO.: 7 encodes SsLsC, SEQ ID NO.: 8 encodes PsLsC, SEQ ID NO.: 9 encodes Dura-PETase, SEQ ID NO.: 10 encodes Thermo-PETase, SEQ ID NO.: 11 encodes Ap-lipase, SEQ ID NO.: 12 encodes Re-lipase, wherein the WT-PETase is originated from Ideonella sakaiensis, the IsGroELS is originated from Ideonella sakaiensis, the IsLsC is originated from Ideonella sakaiensis, the EcGroELS is originated from Escherichia coli, the RsLsC1 and the RsLsC2 is originated from Rhizobacter sp., the SsLsC is originated from Stutzerimonas stutzeri, the PsLsC is originated from Pseudomonas sp., the Dura-PETase is originated from a variant of Ideonella sakaiensis, the Thermo-PETase is originated from a varian of Ideonella sakaiensis, the Ap-lipase is originated from Aquabacterium parvum, the Re-Lipase is originated from Ralstonia eutropha H165.

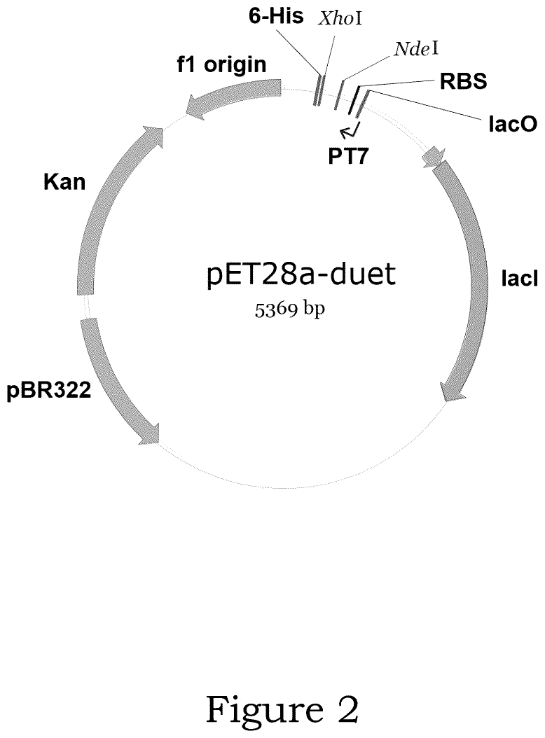

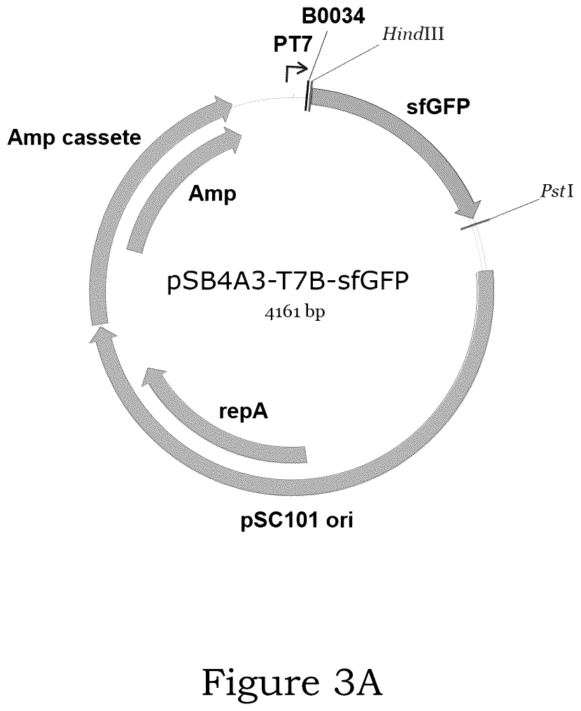

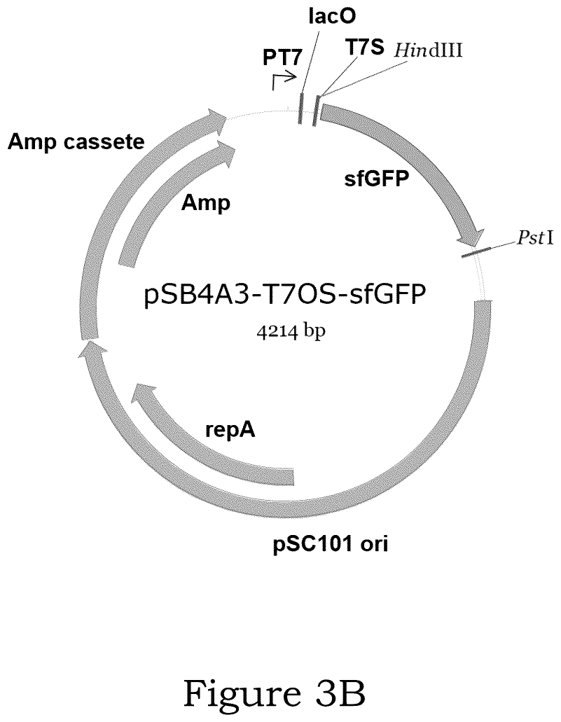

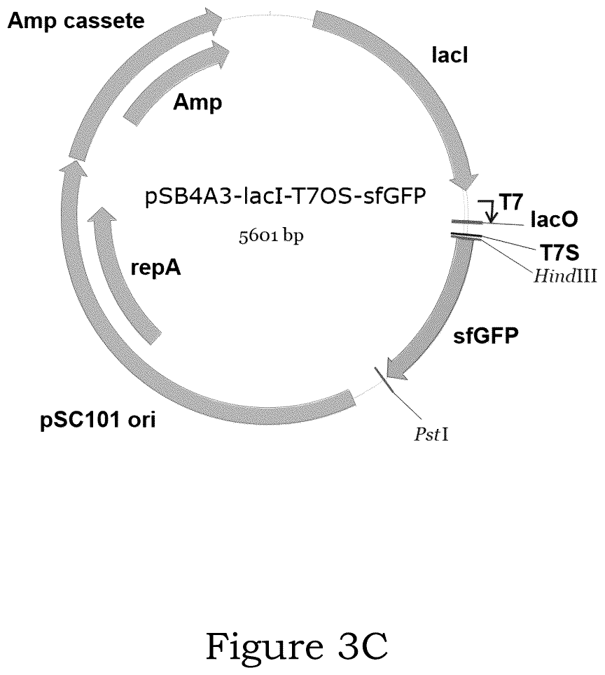

In the specific embodiments, the “clone” refers to integrating the DNA sequence into the plasmid using a restriction enzyme; for example: the restriction enzyme cutting site of the plasmid as shown in FIG. 2 are XhoI and NdeI, the restriction enzyme cutting site of the plasmid as shown in FIGS. 3A, 3B, and 3C are HindIII and PstI respectively.

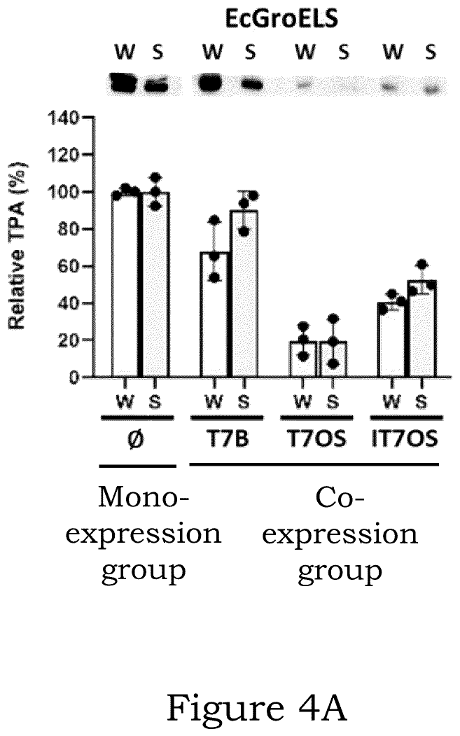

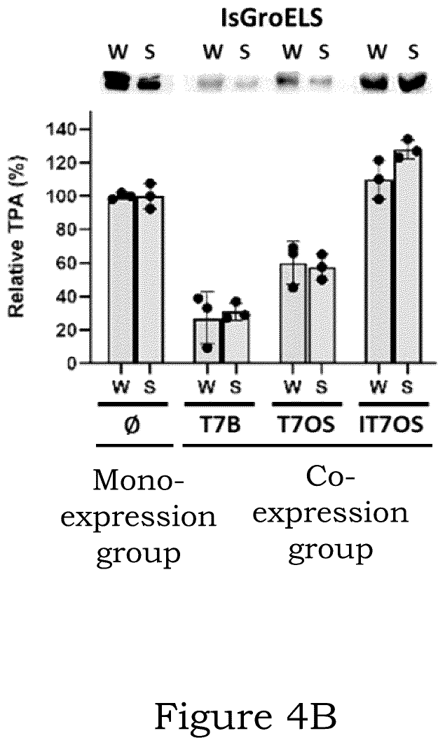

Embodiment 1, comprising steps (A) to (D).
(A) preparing step: preparing E. coli BL21(DE3). Preparing first vector: cloning the sequence as shown in SEQ ID NO.: 1 into the pET28A-duet plasmid as shown in FIG. 2, so as to obtain the first vector. Preparing second vector: cloning the sequence as shown in SEQ ID NO.: 2 into the pSB4A3-T7B-sfGFP plasmid as shown in FIG. 3A, into the pSB4A3-T7OS-sfGFP plasmid as shown in FIG. 3B, and into the pSB4A3-lacI-T7OS-sfGFP plasmid as shown in FIG. 3C respectively, so as to obtain each of the second vector.
(B) introducing and culturing step: co-expression group: introducing the first vector and one of the second vector into the E. coli BL21(DE3) so as to obtain a transformed strain, and the transformed strain is cultured under the culture condition; mono-expression group: introducing the first vector into the E. coli BL21(DE3) so as to obtain another transformed strain, and another transformed strain is cultured under the culture condition.
(C) inducing step: inducing the transformed strain by using isopropyl β-D-1-thiogalactopyranoside (IPTG) (0.1 mM) under the induction condition.
(D) testing step: applying the whole transformed strain interior substance or applying the soluble protein from whole transformed strain interior substance to a plastic under the test condition, wherein the plastic is PET powder, so as to obtain the result as shown in FIG. 4A.

Embodiment 2, comprising steps (A) to (D).
(A) preparing step: preparing E. coli BL21(DE3). Preparing first vector: cloning the sequence as shown in SEQ ID NO.: 1 into the pET28A-duet plasmid as shown in FIG. 2, so as to obtain the first vector. Preparing second vector: cloning the sequence as shown in SEQ ID NO.: 4 into the pSB4A3-T7B-sfGFP plasmid as shown in FIG. 3A, into the pSB4A3-T7OS-sfGFP plasmid as shown in FIG. 3B, and into the pSB4A3-lacI-T7OS-sfGFP plasmid as shown in FIG. 3C respectively, so as to obtain each of the second vector.
(B) introducing and culturing step: Co-expression group: introducing the first vector and one of the second vector into the E. coli BL21(DE3) so as to obtain a transformed strain, and the transformed strain is cultured under the culture condition. Mono-expression group: introducing the first vector into the E. coli BL21(DE3) so as to obtain another transformed strain, and another transformed strain is cultured under the culture condition.
(C) inducing step: inducing the transformed strain by using isopropyl β-D-1-thiogalactopyranoside (IPTG) (0.1 mM) under the induction condition.
(D) testing step: applying the whole transformed strain interior substance or applying the soluble protein from whole transformed strain interior substance to a plastic under the test condition; wherein the plastic is PET powder, so as to obtain the result as shown in FIG. 4B.

From embodiment 1 and embodiment 2, it can be understood that: IsGroELS and EcGroELS affects the protein expression level and the activity of WT-PETase respectively.

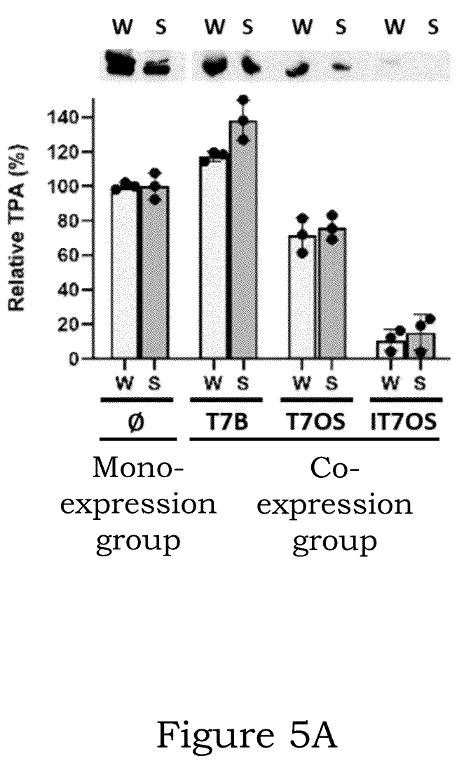

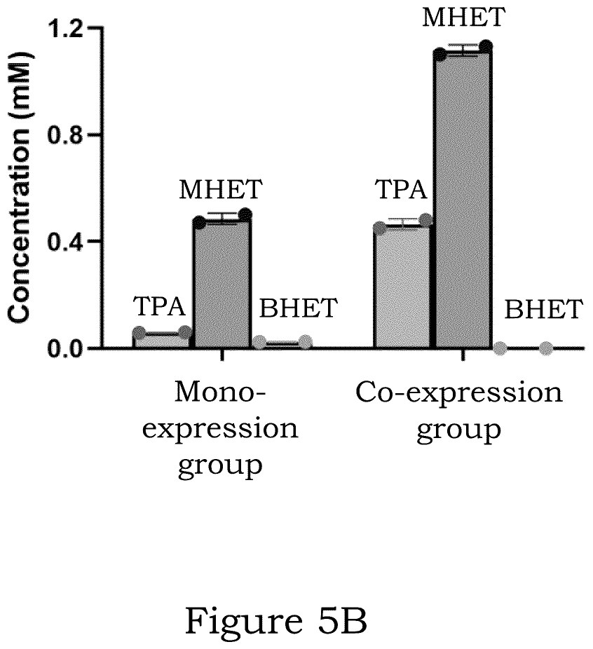

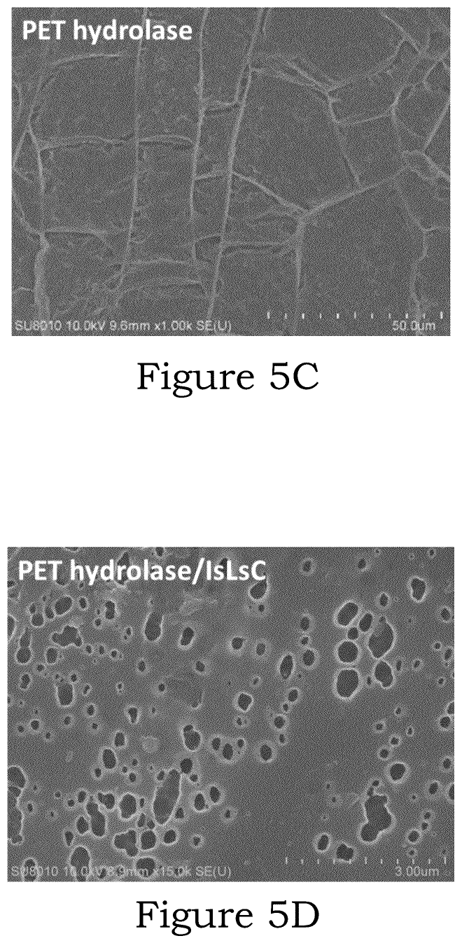

Embodiment 3, comprising steps (A) to (D), wherein step (D) includes step (D1), step (D2), step (D3) or step (D4).
(A) preparing step: preparing E. coli BL21(DE3). Preparing first vector: cloning the sequence as shown in SEQ ID NO.: 1 into the pET28A-duet plasmid as shown in FIG. 2, so as to obtain the first vector. Preparing second vector: cloning the sequence as shown in SEQ ID NO.: 3 into the pSB4A3-T7B-sfGFP plasmid as shown in FIG. 3A, into the pSB4A3-T7OS-sfGFP plasmid as shown in FIG. 3B, and into the pSB4A3-lacI-T7OS-sfGFP plasmid as shown in FIG. 3C respectively, so as to obtain each of the second vector.
(B) introducing and culturing step: co-expression group: introducing the first vector and one of the second vector into the E. coli BL21(DE3) so as to obtain a transformed strain, and the transformed strain is cultured under the culture condition; mono-expression group: introducing the first vector into the E. coli BL21(DE3) so as to obtain another transformed strain, and another transformed strain is cultured under the culture condition.
(C) inducing step: inducing the transformed strain by using isopropyl β-D-1-thiogalactopyranoside (IPTG) (0.1 mM) under the induction condition.
(D1) first testing step: applying the whole transformed strain interior substance or applying the soluble protein from whole transformed strain interior substance to a plastic under the test condition, wherein the plastic is PET powder, so as to obtain the result as shown in FIG. 5A.
(D2) second testing step: applying the whole transformed strain interior substance to a plastic under the test condition, wherein the plastic is PET membrane (2 mg/mL), so as to obtain the result as shown in FIG. 5B, wherein the second vector in the co-expression group is pSB4A3-T7B-sfGFP.
(D3) third testing step: applying the whole transformed strain interior substance to a plastic under the test condition, wherein the plastic is PET membrane (2 mg/mL), so as to obtain the results as shown in FIG. 5C and FIG. 5D.
(D4) fourth testing step: applying the whole transformed strain interior substance to a plastic under the test condition; as shown in Table 1, the plastic of group A is a PET powder mixing with PET powder with 29.5% crystallinity and PET powder with 48% crystallinity, the plastic of group B is a PET powder with 9.4% crystallinity.

TABLE 1

Conversion rate (%)

| | PET | Mono-expression | Co-expression |
|---|---|---|---|
| | (mg/mL) | group IsLsC (−) | group IsLsC (+) |
| Group A | 2.5 | 4.91 | 5.15 |
| | 0.5 | 5.42 | 5.61 |
| | 0.25 | 3.71 | 8.79 |
| | 0.125 | 4.54 | 8.01 |
| Gorup B | 2.0 | 8.23 | 23.9 |
| | 0.4 | 5.75 | 35.3 |
| | 0.2 | 7.93 | 51.7 |
| | 0.1 | 6.35 | 54.1 |

From FIG. 5A, it is observed that, comparing the co-expression group with the mono-expression group, the concentration of TPA is increased in the co-expression group about 40% when the second vector introduced is pSB4A3-T7B-sfGFP. From FIG. 5B, comparing the co-expression group with the mono-expression group, the concentration of TPA is increased in the co-expression group about 8 times, the concentration of MHET is increased in the co-expression group about 2.3 times, wherein the conversion rate is 23.9%. Further, comparing the FIG. 5C with FIG. 5D, as shown in FIG. 5D, it can be obviously observed that the etchings on the PET membrane caused by co-expression group, however, as shown in FIG. 5C, the etchings cannot be observed obviously on the PET membrane.

The results shown in table 1 demonstrate that, better than the mono-expression group, the co-expression group has the ability of degrading PETs with different crystallinity, and especially good at degrading PETs with lower crystallinity. In addition, the co-expression group is prone to degrade the PET with lower concentration, while the mono-expression group is not.

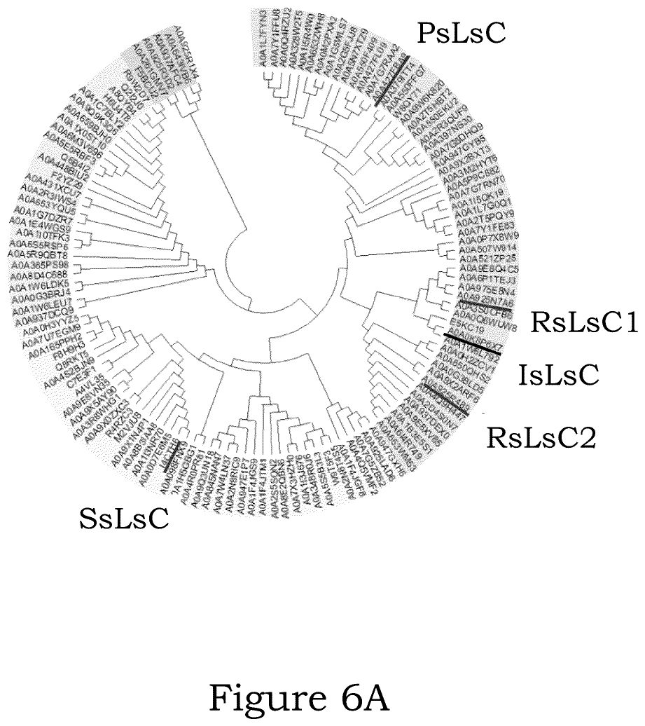

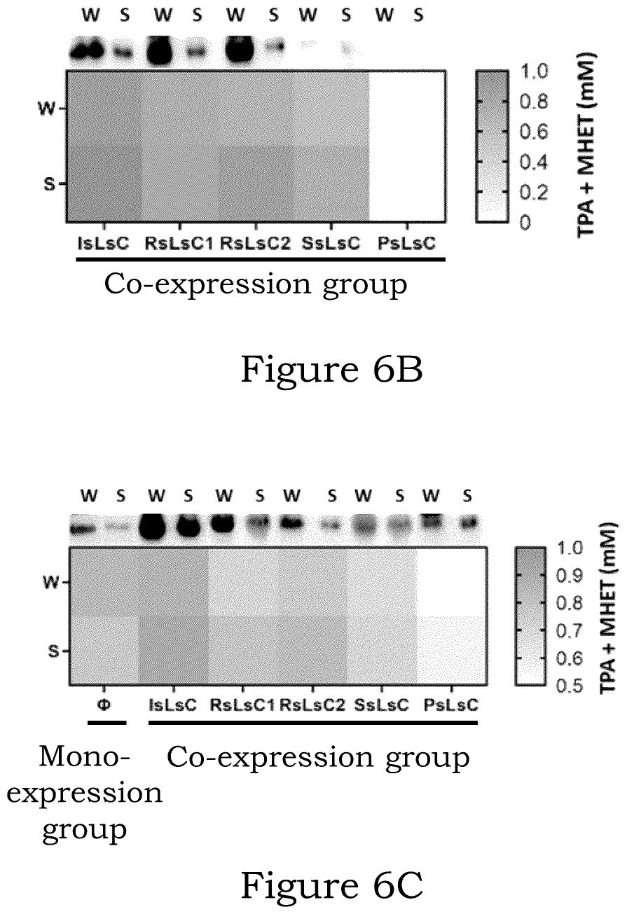

Embodiment 4, comprising steps (A) to (E).
(A) investigating step: according to the sequence as shown in SEQ ID NO.: 3, investigating sequences for homologous proteins through Sequence Similarity Networks (SSNs), so as to obtain the sequence as shown in SEQ ID NO.: 5, the sequence as shown in SEQ ID NO.: 6, the sequence as shown in SEQ ID NO.: 7, the sequence as shown in SEQ ID NO.: 8, and the results of SSNs are shown in FIG. 6A.
(B) preparing step: preparing E. coli BL21(DE3). Preparing first vector: cloning the sequence as shown in SEQ ID NO.: 1 into the pET28A-duet plasmid as shown in FIG. 2, so as to obtain the first vector. Preparing second vector: cloning the sequence as shown in SEQ ID NO.: 5, the sequence as shown in SEQ ID NO.: 6, the sequence as shown in SEQ ID NO.: 7, and the sequence as shown in SEQ ID NO.: 8 into the pSB4A3-T7B-sfGFP plasmid as shown in FIG. 3A respectively, so as to obtain each of the second vector.
(C) introducing and culturing step: co-expression group: introducing the first vector and one of the second vector into the E. coli BL21(DE3) so as to obtain a transformed strain, and the transformed strain is cultured under the culture condition; mono-expression group: introducing the first vector into the E. coli BL21(DE3) so as to obtain another transformed strain, and another transformed strain is cultured under the culture condition.
(D) inducing step: inducing the transformed strain by using isopropyl β-D-1-thiogalactopyranoside (IPTG) (0.1 mM) under the induction condition.
(E) testing step: applying the whole transformed strain interior substance or applying the soluble protein from whole transformed strain interior substance to a plastic under the test condition, wherein the plastic is PET powder, so as to obtain the result as shown in FIG. 6B.

Embodiment 5, comprising steps (A) to (E).

The results of embodiment 5 are shown in FIG. 6C. The difference between embodiment 5 and embodiment 4 is step (C), step (C) in the embodiment 5: Co-expression group: introducing the first vector, one of the second vector, and a third vector into the E. coli BL21(DE3) so as to obtain the transformed strain, wherein the third vector is pRARE.

Referring to related figures to compare embodiment 4 with embodiment 5, in the co-expression group of the embodiment 4, with the second chaperone protein including IsLsC, RsLsC1, RsLsC2, and SsLsC, the PETase shows the ability of degrading the PET; further, the co-expression group of the embodiment 5 introduced the third vector (pRARE), the protein expression level of soluble protein comprising PETase and second chaperone protein including IsLsC, RsLsC1, RsLsC2, and SsLsC in the transformed strain are elevated, therefore, the PET degrading efficiency of every co-expression group were also elevated.

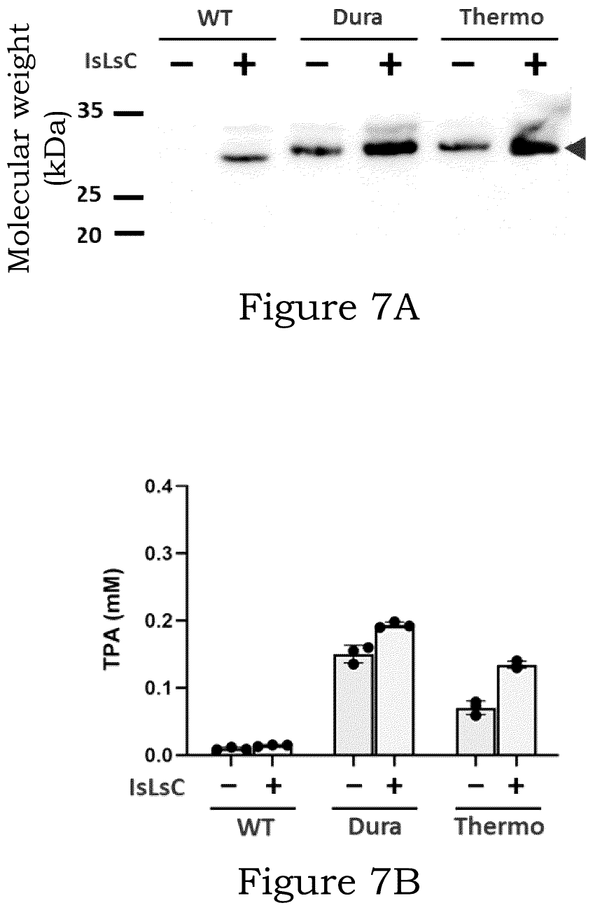

Embodiment 6, comprising steps (A) to (D).
(A) preparing step: preparing E. coli BL21(DE3). Preparing first vector: cloning the sequence as shown in SEQ ID NO.: 1, the sequence as shown in SEQ ID NO.: 9, and the sequence as shown in SEQ ID NO.: 10 into the pET28A-duet plasmid as shown in FIG. 2 respectively, so as to obtain each of the first vector. Preparing second vector: cloning the sequence as shown in SEQ ID NO.: 2 into the pSB4A3-T7B-sfGFP plasmid as shown in FIG. 3A, so as to obtain the second vector.
(B) introducing and culturing step: co-expression group: introducing one of the first vector and the second vector into the E. coli BL21(DE3) so as to obtain a transformed strain, and the transformed strain is cultured under the culture condition; mono-expression group: introducing the first vector into the E. coli BL21(DE3) so as to obtain another transformed strain, and another transformed strain is cultured under the culture condition.
(C) inducing step: inducing the transformed strain by using isopropyl β-D-1-thiogalactopyranoside (IPTG) (0.1 mM) under the induction condition.
(D) testing step: applying the whole transformed strain interior substance to a plastic under the test condition, wherein the plastic is PET powder, so as to obtain the result as shown in FIG. 7A and FIG. 7B.

With reference to FIG. 7A, comparing to mono-expression group, the protein expression levels are upregulated in the co-expression group wherein the PETase including WT-PETase, Dura-PETase, and Thermo-PETase are co-expressed with IsLsC respectively. Further, with reference to FIG. 7B, comparing to mono-expression group, the PET degrading efficiency of the co-expression group are elevated in the co-expression group wherein PETase including WT-PETase, Dura-PETase, and Thermo-PETase are co-expressed with IsLsC respectively; especially, the PET degrading efficiency of the co-expression groups are elevated more compared with WT-PETase when the PETase is Dura-PETase and Thermo-PETase.

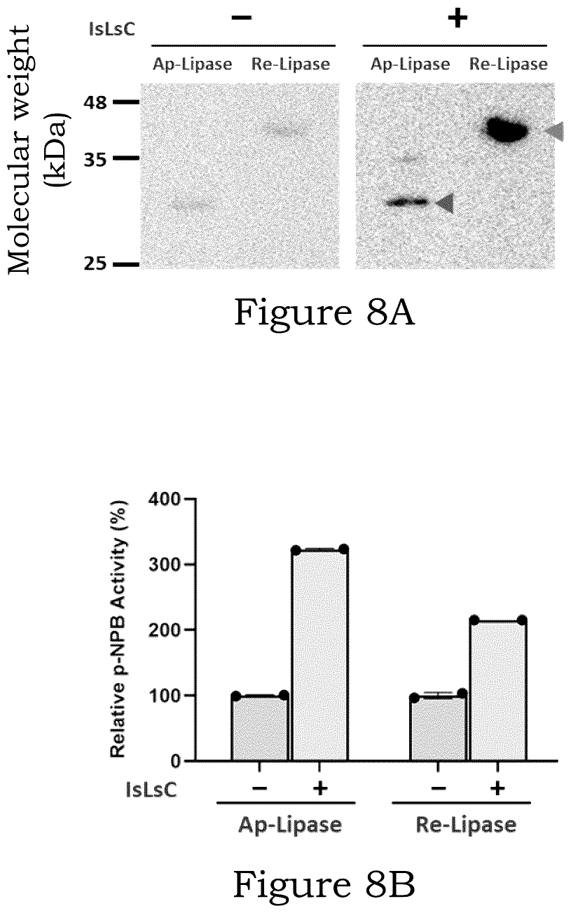

Embodiment 7, comprising steps (A) to (D).
(A) preparing step: preparing E. coli BL21(DE3). Preparing first vector: cloning the sequence as shown in SEQ ID NO.: 11, and the sequence as shown in SEQ ID NO.: 12 into the pET28A-duet plasmid as shown in FIG. 2 respectively, so as to obtain each of the first vector. Preparing second vector: cloning the sequence as shown in SEQ ID NO.: 2 into the pSB4A3-T7B-sfGFP plasmid as shown in FIG. 3A, so as to obtain the second vector.
(B) introducing and culturing step: co-expression group: introducing one of the first vector and the second vector into the E. coli BL21(DE3) so as to obtain a transformed strain, and the transformed strain is cultured under the culture condition; mono-expression group: introducing the first vector into the E. coli BL21(DE3) so as to obtain another transformed strain, and another transformed strain is cultured under the culture condition.
(C) inducing step: inducing the transformed strain by using isopropyl β-D-1-thiogalactopyranoside (IPTG) (0.1 mM) under the induction condition.
(D) testing step: applying the whole transformed strain interior substance to 4-Nitrophenol (p-NPB) under the test condition, so as to obtain the result as shown in FIG. 8A and FIG. 8B.

With reference to FIG. 8A and FIG. 8B, comparing to mono-expression group, the protein expression levels are upregulated in the co-expression group, wherein the lipase including Ap-lipase, and Re-lipase are co-expressed with IsLsC respectively. Further, the activity of the co-expression groups is greater comparing with mono-expression groups respectively. The result indicates that, as well as transformed strain co-expressing IsLsC with the PETase, the protein expression level and substrate degrading activity are elevated when the transformed strain co-expressing IsLsC with the lipase; in other words, the lipase secretion chaperone protein IsLsC not only promotes the protein expression level and substrate degrading activity of the PETase and the lipase, but an esterase or a hydrolase.

The disclosure has been described above are just some preferred embodiments of the present invention, and should not be used for limiting the claims of the present invention; In other words, any modifications and similar arrangements based on the present invention, should be included and protected by the claim of the present invention.

Sequence listing:

<ST26SequenceListing dtdVersion = ″V1_3″ fileName = ″PI-112-159-US.xml″
softwareName = ″WIPO Sequence″ software Version = ″2.3.0″
productionDate = ″2024-11-01″>
<ApplicantFileReference>PI-112-159-US</ApplicantFileReference>
<EarliestPriority ApplicationIdentification>
<IPOfficeCode>US</IPOfficeCode>
<ApplicationNumberText>63/547,394</ApplicationNumberText>
<FilingDate>2023-11-06</FilingDate>
</EarliestPriority ApplicationIdentification>
<ApplicantName languageCode = ″zh″> [symbol] </ApplicantName>
<ApplicantNameLatin>National Cheng Kung University</ApplicantNameLatin>
<InventionTitle languageCode = ″en″>TRANSFORMED STRAIN AND
METHOD FOR DEGRADING PET BY USING THE SAME</InventionTitle>
<SequenceTotalQuantity>12</SequenceTotalQuantity>
<SequenceData sequenceIDNumber = ″1″>
<INSDSeq>
<INSDSeq_length>873</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..873</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q26″>
<INSDQualifier_name>organism</INSDQualifier_name>
<INSDQualifier_value>Ideonella sp.</INSDQualifier_value>
</INSDQualifier>
</INSDFeature_quals>
</INSDFeature>
</INSDSeq_feature-table>
<INSDSeq_sequence>atgaactttccccgcgcttcccgcctgatgcaggccgccgttctcggcg
ggctgatggccgtgtcggccgccgccaccgcccagaccaacccctacgcccgcggcccgaacccgacagccgcctc
actcgaagccagcgccggcccgttcaccgtgcgctcgttcaccgtgagccgcccgagcggctacggcgccggcacc
gtgtactaccccaccaacgccggcggcaccgtgggcgccatcgccatcgtgccgggctacaccgcgcgccagtcga
gcatcaaatggtggggcccgcgcctggcctcgcacggcttcgtggtcatcaccatcgacaccaactccacgctcgacc
agccgtccagccgctcgtcgcagcagatggccgcgctgcgccaggtggcctcgctcaacggcaccagcagcagccc
gatctacggcaaggtcgacaccgcccgcatgggcgtgatgggctggtcgatgggcggtggcggctcgctgatctcgg
cggccaacaacccgtcgctgaaagccgcggcgccgcaggccccgtgggacagctcgaccaacttctcgtcggtcacc
gtgcccacgctgatcttcgcctgcgagaacgacagcatcgccccggtcaactcgtccgccctgccgatctacgacagca
tgtcgcgcaatgcgaagcagttcctcgagatcaacggtggctcgcactcctgcgccaacagcggcaacagcaaccag
gcgctgatcggcaagaagggcgtggcctggatgaagcgcttcatggacaacgacacgcgctactccaccttcgcctgc
gagaacccgaacagcacccgcgtgtcggacttccgcaccgcgaactgcagctga</INSDSeq_sequence>
</INSDSeq>
</SequenceData>
<SequenceData sequenceIDNumber = ″2″>
<INSDSeq>
<INSDSeq_length>1647</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..1647</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q2″>
<INSDQualifier_name>organism</INSDQualifier_name>
<INSDQualifier_value>Ideonella sp.</INSDQualifier_value>
</INSDQualifier>
</INSDFeature_quals>
</INSDFeature>
</INSDSeq_feature-table>
<INSDSeq_sequence>atgatggcagcaaaagacgtggttttcggcggtgatgcgcgcgcgcg
catggtcgaaggcgtgaacatcctggccaacgccgtcaaggtcaccctcggcccgaagggccgcaacgtggtgctgg
agcgctcgttcggcgcccccaccgtcaccaaggacggtgtctcggtcgccaaggagatcgagctcaaggacaagctg
cagaacatgggcgcccagatggtcaaggaagtcgcttccaagaccagcgacaacgccggtgacggcacgacgaccg
ccaccgtgctggcccaggccatcgtgcgcgaaggcatgaagtacgtggccgccggcatgaacccgatggacctgaag
cgcggcatcgaccgtgccgtcgccgcgctggtcgagcagttgaagaaggcctcgaaggcgacgacgacctcgaagg
aaatcgcccaggtcggcgcgatctcggccaacagcgacgagtcgatcggcaagatcatcgccgacgcgatggacaa
ggtcggcaaggaaggcgtcatcaccgtcgaggacggcaagtcgctggacaacgagctggacgtcgtcgaaggcatg
cagttcgaccgcggctacctgtcgccctacttcatcaacaacccggacaagcaggctgccatcctggacaacccgttcgt
cctgctgttcgacaagaagatcagcaacatccgcgacctgctgccgacgctggaagccgtcgccaaggccggccgtc
cgctgctgatcatcgccgaggaagtcgaaggcgaggcgctggcgaccctggtggtcaacaccatccgcggcatcctg
aaggtcgccgccgtcaaggcgcccggcttcggtgaccgccgcaaggccatgctggaggacatcgcgatcctgaccg
gcggcaaggtgatcgccgaggaagtgggcctgtcgctcgagaaggtcacgctggccgacctgggccaggccaagcg
cgtcgaggtggcgaaggaaaacaccacgatcatcgacggcgccggcgccgcggccgacatcgaggcccgcgtcaa
gcagatccgtgtccagatcgaggaagccaccagcgactacgaccgcgagaagctgcaggagcgtgtcgcgaagctg
gccggcggcgtggccgtcatccgcgtcggtgccgccaccgaagtcgagatgaaggagaagaaggcccgcgtcgaa
gacgcgctgcacgccacgcgtgccgccgtcgaggaaggcatcgtcgccggtggtggcgtcgcgctgctgcgtgcga
agcagtcggcgggcgagatcaagggcgacaacgccgaccaggacgccggcatcaagctggtgctgaaggcgatcg
aggccccgctgcgcgagatcgtctacaacgccggtggcgaagcctccgtcgtcgtgaacgccgtgctggccggcaag
ggcaactacggcttcaacgccgccaacgacacctatggcgacatgatcgagatgggcatcctggacccgaccaaggt
gacccgcaccgcgctgcagaacgccgcgtcggtggcctcgctgatgctgaccaccgagtgcatggtcgccgaggcg
ccgaaggacgaggcgcccgccccggccggcggcatgggcggcatgggtggcatgggcggcatggacatgtga
</INSDSeq_sequence>
</INSDSeq>
</SequenceData>
<SequenceData sequenceIDNumber = ″3″>
<INSDSeq>
<INSDSeq_length>621</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..621</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q6″>
<INSDQualifier_name>organism</INSDQualifier_name>
<INSDQualifier_value>Ideonella sp.</INSDQualifier_value>
</INSDQualifier>
</INSDFeature_quals>
</INSDFeature>
</INSDSeq_feature-table>
<INSDSeq_sequence>atgctgcgtggacctgccaatgaacccactggcgcctctgcggcggca
gccagtagctcggtcatggtggccgatgcttcgggcgcaaacctggacgcggcgcgcttatttgagttgggattcgcgg
gtgggctggtgattgatcgcgatacacgggcagtcattgaggcagtattaaattcgatgccggaacagccctcggctgat
gacctgcaacgactggagcgtacacttcgtgaaggactgcctcgtgaagatgcggagcgagctttaaaattatttggaag
ctatcgcgattatactgcggacgtgcggcgacaaatggaacccctgggggttccgcgtaatctgcaagaaatgaatgcat
tctttgatcagatggaggcgattaaacaacgtcatttcgacgcggcgaccgcgcaagcgctgtttggaccggcggatatg
catgctcgtgtgagtatggaagccatgtttgttgatcaggatccatctttaaccttagagcagaaaaaacagcggctcgatg
agctcagagcaaagctgccgccggatcaacgtagcctgatccctgaaccatcccagccggcctcactcgagcaccacc
accaccaccactga</INSDSeq_sequence>
</INSDSeq>
</SequenceData>
<SequenceData sequenceIDNumber = ″4″>
<INSDSeq>
<INSDSeq_length>1647</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..1647</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q8″>
<INSDQualifier_name>organism</INSDQualifier_name>
<INSDQualifier_value>Escherichia coli</INSDQualifier_value>
</INSDQualifier>
</INSDFeature_quals>
</INSDFeature>
</INSDSeq_feature-table>
<INSDSeq_sequence>atggcagctaaagacgtaaaattcggtaacgacgctcgtgtgaaaatgc
tgcgcggcgtaaacgtactggcagatgcagtgaaagttaccctcggtccaaaaggccgtaacgtagttctggataaatct
ttcggtgcaccgaccatcaccaaagatggtgtttccgttgctcgtgaaatcgaactggaagacaagttcgaaaatatgggt
gcgcagatggtgaaagaagttgcctctaaagcaaacgacgctgcaggcgacggtaccaccactgcaaccgtactggct
caggctatcatcactgaaggtctgaaagctgttgctgcgggcatgaacccgatggacctgaaacgtggtatcgacaaag
cggttaccgctgcagttgaagaactgaaagcgctgtccgtaccatgctctgactctaaagcgattgctcaggttggtacca
tctccgctaactccgacgaaaccgtaggtaaactgatcgctgaagcgatggacaaagtcggtaaagaaggcgttatcac
cgttgaagacggtaccggtctgcaggacgaactggacgtggttgaaggtatgcagttcgaccgtggctacctgtctcctt
acttcatcaacaagccggaaactggcgcagtagaactggaaagcccgttcatcctgctggctgacaagaaaatctccaa
catccgcgaaatgctgccggttctggaagctgttgccaaagcaggcaaaccgctgctgatcatcgctgaagatgtagaa
ggcgaagcgctggcaactctggttgttaacaccatgcgtggcatcgtgaaagtcgctgcggttaaagcaccgggcttcg
gcgatcgtcgtaaagctatgctgcaggatatcgcaaccctgactggcggtaccgtgatctctgaagagatcggtatggag
ctggaaaaagcaaccctggaagacctgggtcaggctaaacgtgttgtgatcaacaaagacaccaccactatcatcgatg
gcgtgggtgaagaagctgcaatccagggccgtgttgctcagatccgtcagcagattgaagaagcaacttctgactacga
ccgtgaaaaactgcaggaacgcgtagcgaaactggcaggcggcgttgcagttatcaaagtgggtgctgctaccgaagt
tgaaatgaaagagaaaaaagcacgcgttgaagatgccctgcacgcgacccgtgctgcggtagaagaaggcgtggttg
ctggtggtggtgttgcgctgatccgcgtagcgtctaaactggctgacctgcgtggtcagaacgaagaccagaacgtggg
tatcaaagttgcactgcgtgcaatggaagctccgctgcgtcagatcgtattgaactgcggcgaagaaccgtctgttgttgc
taacaccgttaaaggcggcgacggcaactacggttacaacgcagcaaccgaagaatacggcaacatgatcgacatgg
gtatcctggatccaaccaaagtaactcgttctgctctgcagtacgcagcttctgtggctggcctgatgatcaccaccgaatg
catggttaccgacctgccgaaaaacgatgcagctgacttaggcgctgctggcggtatgggcggcatgggtggcatggg
cggcatgatgtaa</INSDSeq_sequence>
</INSDSeq>
</SequenceData>
<SequenceData sequenceIDNumber = ″″>
<INSDSeq>
<INSDSeq_length>645</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..645</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q10″>
<INSDQualifier_name>organism</INSDQualifier_name>
<INSDQualifier_value>Rhizobacter sp.</INSDQualifier_value>
</INSDQualifier>
</INSDFeature_quals>
</INSDFeature>
</INSDSeq_feature-table>
<INSDSeq_sequence>atgcaagcggcggcgaaagcgggtcggtcaccagcttccggtgctgg
cggggcagccccttgggtggtgaccgttagtgatgcaatgggcgcaactatggatgcatctcgcttatttgaattgggcttt
gcgggcgggcttgtcattgataaggacactcgtgcatcagtcgaagcattgctgaatagtatgccggaggatctttctgaa
acagatctggctcgtctggagcgtaccttacgtgaaggtctgccaaaggaagatgccgaaaaagcgtacaagttaatca
ctgactaccgcagttacacaaaggacattcgcgacgagatgcaaccaaaaggcatcccaggtaatatgcaggaggcgc
gcacttttttcgaccagatggaagcagtaaagcgtcgtcacttcgacgaagccacggcaaacgctttgtttgggcaagct
gacttttacgcacgtttgtcgatggaagctatgttcgtgcaacaagacactacattgaccgatgaacaaaaaaaggcgcaa
cttgatgctttgcgtgctcagctgcccgcagatcagaaaagtttaattccacaggacggtcccccaccggggcatcgca
acccgcatcactcgagcaccaccaccaccaccactaa
</INSDSeq_sequence>
</INSDSeq>
</SequenceData>
<SequenceData sequenceIDNumber = ″6″>
<INSDSeq>
<INSDSeq_length>594</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..594</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q12″>
<INSDQualifier_name>organism</INSDQualifier_name>
<INSDQualifier_value>Rhizobacter sp.</INSDQualifier_value>
</INSDQualifier>
</INSDFeature_quals>
</INSDFeature>
</INSDSeq_feature-table>
<INSDSeq_sequence>atgagccaacccgcttcgacaggtgatagcaagttagttgaaattgattt
gcttgggacaaaactggacgccgcccgcttgttcgacttaggcttcgctggtggattgaacattgatcaacatacccgctc
gacgttggacaccctgctgatgaacatgtctgacacacccgcggcacaagagatcgagaaattggaatggaccttgcgt
aacggattgccgaaggatgacgccgagaaggcaattaaaatgtttcacggctatcgtgcatacctgggtgacatgaaag
gtgagttgcagcgcatgggtattcccgagactccagcggcagctaacgcgtacttcgatcaactggcgcttatgcagcg
ccgccatttcgatgacacaaccgcagcggctttgtttgggcaggaaaatcagaacgcacgtcttgtcatgcaagcagctc
ttatcacccaaaatgaagcgttatcagccagcgagaagaaggaacaacttgacttattacgcacccagttgcccgagggt
aaacgtgatcttatcccagcgaccgaaccagccaaacctctcgagcaccaccaccaccaccactaa
</INSDSeq_sequence>
</INSDSeq>
</SequenceData>
<SequenceData sequenceIDNumber = ″7″>
<INSDSeq>
<INSDSeq_length>1032</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..1032</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q14″>
<INSDQualifier_name>organism</INSDQualifier_name>
<INSDQualifier_value>unidentified</INSDQualifier_value>
</INSDQualifier>
</INSDFeature_quals>
</INSDFeature>
</INSDSeq_feature-table>
<INSDSeq_sequence>atgtcgaaatacataatattacttgttctggctgtggccctttcacttacaat
tatgcaaagtcgtagcactccaggtcccgctgcggtagcagagtcgccgtcagcgacttcccctgcaacaacagccccc
acaccaccccgtcaacctttgaaagcgcagccggaaccccaaggaaccccgcagttaccgaccagcttcaaaggaac
ccaggtagatggacaattccgcctggacgaagccggcaaccttttaattggagcggaattacgccatctgtttgattacttc
ttagcagcagcaggagaagaacctcttaaaaactcgattgagcgtcttcgtcgttatattgtcgctgagttaccagaaccg
gctcaaagtcaagcctcggctgtattaacacagtaccttaattataaacgccagcttttggatgtagaagcaacttattcacg
catcaccgatatttcagctttgcgccagcgcctgagcgccgtacaggcgttacgcgcgcgcgtgttagaacccgcagtc
caccaggctttctttgctttggacgaagcctatgatcgctttagcctggaacgcttagctattcaagcagactcagcattaga
tagcgaagcaaagggtcgtgcgatcgaccagctgcgcgccggattaccaggggaccttcaggaacttctggttccaca
attacagtcggagttgcgtgagcaaacagttgctttgcaggcccaaggtgcaaatgctcagcaaatccgccagctgcgc
caacagttggtggggtcagaagccgccacccgcttggaggcattggaccgtcagcgtgaacaatggcagcagcgcgtt
gcagtttatcgtcaagaacgtcagcgtattgaagcaacgcgcggattggacgacgtggagcgccgctctgctatcgaac
gccttgaggcggagcaattcgacgagggagaacgtctgcgccttgtcgcagcgtttcagcaacaggaagttgcagagc
gccaccaccaccaccaccactaa</INSDSeq_sequence>
</INSDSeq>
</SequenceData>
<SequenceData sequenceIDNumber = ″8″>
<INSDSeq>
<INSDSeq_length>1083</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..1083</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q16″>
<INSDQualifier_name>organism</INSDQualifier_name>
<INSDQualifier_value>Pseudomonas sp.</INSDQualifier_value>
</INSDQualifier>
</INSDFeature_quals>
</INSDFeature>
</INSDSeq_feature-table>
<INSDSeq_sequence>atgagtaaattctttagtctttcattggtcgcagtagtggtagctggaggat
tgactctgtattggcgctggcccgcggcagtccccgaggtgctgcccaccgctgcggttgcagtgcccagtcgtcagac
gtccactgagcccctttcagttcctatcgccaaagcctcatccgaggctcaaccgtcagtagcagaaagccttccctctct
ggtggacacagaagttgatgggcagttacgtacagacgcagccgggaatttagtgttagacttagcagttcgtgattatttt
gactactttcttagcgcggtggatcactccggccttgatgcagtaatcgaagcgttacttgccgacgctggacgtcgccttc
ccgagcctgctcttggccaaatgatctctttgcttggagattatctggactataaacgcgccagtatggctttgatgcagca
gccattggacgcgcaacagcaggtcgaacccaaagcacaattacaggctttgcagtctgcatttgctcgcctggacgaat
tgcgccgtgcccatttctccgcagccgcgcaagaggcgttgttcggagcagagcaggcatacgctcgctacaccttaga
ttctcttgctttacaacaacgtgacgatcttggcgaggcgcaacgtgcgcagcgtctggaacagcttcgcgaacgtcttcc
cgaggcattgcgtgaatctgagcagcgtcaacaactggcgttggagcaacttcaacgtagcgagcaactttggcgtgat
ggcgctgacgagcaacaagtacgtgagttcttggcgatgacctatgaccctcagactgtacagcgtttactggacgagca
acgtcgcgaacgtgattggcagcaacattatcaggcttaccgcaacgagctggcttctttgcaagggcgcggtttatctga
agctgatggtgagcagcttcagcgtcaacttcgcgaacgccttttttcgtcagaagatcgtcatcgtgtggaaacgtacgat
gctatcgctgcaaaacagccagaaccattggaccatctcgagcaccaccaccaccaccactaa</INSDSeq_sequence>
</INSDSeq>
</SequenceData>
<SequenceData sequenceIDNumber = ″9″>
<INSDSeq>
<INSDSeq_length>801</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..801</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q28″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>atgcagaccaatccgtatgcgcgcggtccgaatccgacagccgccagt
ttggaagcgagcgctggtccattcaccgttcgctcctttaccgtgagtagaccgagcggttatggcgctggcaccgtttac
tatccaacaaatgctgggggtaccgtgggcgccatagccatagttcccgggtatacggcacggcagtcatcaattaaatg
gtggggaccgcgtctggcatcccacggtttogtagtaattacaattgacacaaattccacgtttgactatccatcaagtcgg
agttcgcaacaaatggccgcgctgcgccaggtggcgtcgttaaacggtgacagtagcagcccgatttacggaaaggtc
gataccgctcgtatgggtgttatggggcatagtatgggaggtggagcatccctgcgatctgctgctaacaacccttcgctg
aaagcagcgattcctcaagcaccatgggattctcaaacaaattttagttctgtaactgtgcccacgctgatcttcgcatgtga
aaacgatagtatagccccggtcaactctcatgcacttcctatctatgattctatgtcacgcaacgctaagcagtttctcgaaa
ttaatggtggctcacattcctgtgcgaatagcggcaattctaaccaagcattaatcggaaaaaaaggcgttgcatggatga
aacgttttatggacaatgatactaggtattctacttttgcctgcgagaacccgaatagcaccgcagtgtctgattttcgtacag
cgaattgcagcctcgagtga</INSDSeq_sequence>
</INSDSeq>
</SequenceData>
<SequenceData sequenceIDNumber = ″10″>
<INSDSeq>
<INSDSeq_length>801</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..801</INSDFeature_location>
<INSDFeature_quals>
<INSDQualifier>
<INSDQualifier_name>mol_type</INSDQualifier_name>
<INSDQualifier_value>other DNA</INSDQualifier_value>
</INSDQualifier>
<INSDQualifier id = ″q30″>
<INSDQualifier_name>organism</INSDQualifier_name>
<INSDQualifier_value>synthetic construct</INSDQualifier_value>
</INSDQualifier>
</INSDFeature_quals>
</INSDFeature>
</INSDSeq_feature-table>
<INSDSeq_sequence>atgcagaccaatccgtatgcgcgcggtccgaatccgacagccgccagt
ttggaagcgagcgctggtccattcaccgttcgctcctttaccgtgagtagaccgagcggttatggcgctggcaccgtttac
tatccaacaaatgctgggggtaccgtgggcgccatagccatagttcccgggtatacggcacggcagtcatcaattaaatg
gtggggaccgcgtctggcatcccacggtttcgtagtaattacaattgacacaaattccacgttagaccagccagaaagtc
ggagttcgcaacaaatggccgcgctgcgccaggtggcgtcgttaaacggtacaagtagcagcccgatttacggaaagg
tcgataccgctcgtatgggtgttatggggtggagtatgggaggtggaggctccctgatctctgctgctaacaacccttcgc
tgaaagcagcggcgcctcaagcaccatggcactcttcgacaaattttagttctgtaactgtgcccacgctgatcttcgcatg
tgaaaacgatagtatagccccggtcaactcttcagcacttcctatctatgattctatgtcacgcaacgctaagcagtttctcg
aaattaatggtggctcacattcctgtgcgaatagcggcaattctaaccaagcattaatcggaaaaaaaggcgttgcatgga
tgaaacgttttatggacaatgatactaggtattctacttttgcctgcgagaacccgaatagcaccgcagtgtctgattttcgta
cagcgaattgcagcctcgagtga</INSDSeq_sequence>
</INSDSeq>
</SequenceData>
<SequenceData sequenceIDNumber = ″11″>
<INSDSeq>
<INSDSeq_length>1224</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..1224</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q32″>
<INSDQualifier_name>organism</INSDQualifier_name>
<INSDQualifier_value>Aquabacterium parvum</INSDQualifier_value>
</INSDQualifier>
</INSDFeature_quals>
</INSDFeature>
</INSDSeq_feature-table>
<INSDSeq_sequence>atgggcagcagccatcatcatcatcatcacagcagcggcctggtgccg
cgcggcagccatatggctagcatgactggtggacagcaaatgggtcgcggatccgaattcaagcttatgaggagcgat
gccttctacacgccgccccagccgttgccgagcgggccccatggcacgctgattcgctcccgtccattcattccggttgg
attgctggcttacggctggcaggtcatgtacaagtccaacgacgtcaatggcttgccgattgccatcaccggcaccgtgc
tggtgccctttacgccatggctctccggcccccggccggttattgcgtgggctccaggaacgcagggccaggccgaca
actgcgcgccctcgcaccagtacgcgctgagcacggaatatgaagcgacgattgccggcgcgacgccggcaaccctc
gcccgcggttgggcgatcgcgatgacggactatcagggtcttggtacaccgggcgatcacacctgggggatcggcaa
ggccgaagggcaagccgtcctcgacgcggcactggcggcgcagcagttgagcggggcgggcctgtcggcgaacgc
cggtctgaatgtgcggggcgttgccgcaggagctgggccattcagcatcgctgatgtatatgcgaacgttaatggtggcc
cgttcggcggggtagggccgattgcgctgatgggcctgaatgccgcctaccctgaacttggcctctcatcgatcctcaca
ccctatgggcagtcggtgattgccgactttcgcggcacaaaatgcctgcttgacgtgactgccagctatccgttcctgtctg
actcggtcctcgtccaggctccagccatcctgaaccgcgccgactggctggcgcgcatggcgcagaaccagagcggc
aatggcgtgcctgccgtaccggcattggtgtacgcgggaaccctcgacgagatcatcccctttgggcagaaccagaag
ctgtttgccgcctggtgtgcgcgcggtggcaacgtggcgttccggaccatcggcgcgaccgaacacgccactgggctg
cttctcggcttgccggttgcactggactggatgacacagcgattcggcagtgtccctgcggtatccgagtgcccatag
</INSDSeq_sequence>
</INSDSeq>
</SequenceData>
<SequenceData sequenceIDNumber = ″12″>
<INSDSeq>
<INSDSeq_length>840</INSDSeq_length>
<INSDSeq_moltype>DNA</INSDSeq_moltype>
<INSDSeq_division>PAT</INSDSeq_division>
<INSDSeq_feature-table>
<INSDFeature>
<INSDFeature_key>source</INSDFeature_key>
<INSDFeature_location>1..840</INSDFeature_location>
<INSDFeature_quals>
<INSDQualifier>
<INSDQualifier_name>mol_type</INSDQualifier_name>
<INSDQualifier_value>genomic DNA</INSDQualifier_value>
</INSDQualifier>
<INSDQualifier id = ″q34″>
<INSDQualifier_name>organism</INSDQualifier_name>
<INSDQualifier_value>Ralstonia sp.</INSDQualifier_value>
</INSDQualifier>
</INSDFeature_quals>
</INSDFeature>
</INSDSeq_feature-table>
<INSDSeq_sequence>atgaaaaatgctccgatccgttcagttttggctacttgtttggttgctgctg
ctgcttctgctttggctcaaactactgctccaacttctgcttctttgaatgcttctgctggtccattgtctgtttctactgcttctgtt
actgctccaagaggttttggtggtggtactatttattatccaactactcaagctagatatggtgttattgctatttctccaggtttt
actgctactcaaatttctattgcttggttgggtagaagaattgctactcatggttttgttgttattactttgaatactttgactacttt
ggatcaaccagattctagagctaatcaattgatggctgctttgaatcatgttattaattcttcttcttctgctgttaaagctagaat
tgatacttctagattggctgttgctggtcattctatgggtggtggtggtgctttgattgctgctagagataatccaactttgaaa
gcttcttatccattgactccatggaatattaattctgcttttactactgttagagttccaactatgattgttggtgctgatggtgat
actattgctccagttggtactcatgctagaccattttatgctgctttgccaggtgctacttctaaagcttatggtgaattgaatg
gtgctactcatttttctccaaattctactaatactccaattggtagatatggtgttgcttggatgaaaagatttttggatggtgata
ctagatattctacttttttgtgtggtactgaacatactagatatgctactgctctggtgttcgaccgctataatcagaactgccc
atattga</INSDSeq_sequence>
</INSDSeq>
</SequenceData
</ST26SequenceListing> [symbol]
indicates data missing or illegible when filed

What is claimed is:

1. A transformed strain, comprising:
a host cell;
a first nucleotide sequence located inside the host cells, wherein the first nucleotide sequence encoding a PETase originated from Ideonella; and
a second nucleotide sequence located inside the host cell, wherein the second nucleotide comprising a first chaperone nucleotide sequence or a second chaperone nucleotide sequence; wherein:
the first chaperone nucleotide sequence encodes a molecular chaperone protein (GroELS), the second chaperone nucleotide sequence encodes a lipase secretion chaperone protein (LsC), and at least one of the first nucleotide sequence and the second nucleotide sequence is exogenous.

2. The transformed strain as claimed in claim 1, wherein the host cell is Escherichia coli.

3. The transformed strain as claimed in claim 1, wherein the PETase is further originated from Escherichia, Pichia, Bacillus, Pseudomonas, Stutzerimonas, Streptomyces, Aquabacterium, Ralstonia, or Rhizobacter.

4. The transformed strain as claimed in claim 1, wherein the molecular chaperone protein is originated from Escherichia, Ideonella, Pichia, Bacillus, Pseudomonas, Stutzerimonas, Streptomyces, Aquabacterium, Ralstonia, or Rhizobacter.

5. The transformed strain as claimed in claim 1, wherein the lipase secretion chaperone protein is originated from Escherichia, Ideonella, Pichia, Bacillus, Pseudomonas, Stutzerimonas, Streptomyces, Aquabacterium, Ralstonia, or Rhizobacter.

6. The transformed strain as claimed in claim 1, wherein the first nucleotide sequence comprises: a nucleotide sequence having at least 70% sequence identity to SEQ ID NO.: 1 and same activity with the PETase, a nucleotide sequence having at least 70% sequence identity to SEQ ID NO.: 9 and same activity with the PETase, or a nucleotide sequence having at least 70% sequence identity to SEQ ID NO.: 10 and same activity with the PETase.

7. The transformed strain as claimed in claim 1, further comprising a first vector and a second vector, the first vector and the second vector are located inside the host cell respectively, wherein:
the first nucleotide sequence is integrated at the first vector, the second nucleotide sequence is integrated at the second vector, and the first vector is different from the second vector.

8. The transformed strain as claimed in claim 7, wherein the first vector has a first promoter, the second vector has a second promoter, the first promoter is identical to the second promoter.

9. The transformed strain as claimed in claim 8, wherein the first vector is pET28a, the second vector is pSB4A3.

10. The transformed strain as claimed in claim 8, wherein the first promoter is T7 promoter, the second promoter is T7 promoter.

11. The transformed strain as claimed in claim 1, wherein the first chaperone nucleotide sequence comprises: a nucleotide sequence having at least 70% sequence identity to SEQ ID NO.: 2 and same activity with the molecular chaperone protein, or a nucleotide sequence having at least 70% sequence identity to SEQ ID NO.: 4 and same activity with the molecular chaperone protein.

12. The transformed strain as claimed in claim 1, wherein the second chaperone nucleotide sequence comprises: a nucleotide sequence having at least 70% sequence identity to SEQ ID NO.: 3 and same activity with the lipase secretion chaperone protein, a nucleotide sequence having at least 70% sequence identity to SEQ ID NO.: 5 and same activity with the lipase secretion chaperone protein, a nucleotide sequence having at least 70% sequence identity to SEQ ID NO.: 6 and same activity with the lipase secretion chaperone protein, a nucleotide sequence having at least 70% sequence identity to SEQ ID NO.: 7 and same activity with the lipase secretion chaperone protein, a nucleotide sequence having at least 70% sequence identity to SEQ ID NO.: 8 and same activity with the lipase secretion chaperone protein.

13. The transformed strain as claimed in claim 1, further comprising a third vector, located inside the host cell, used for increasing a tRNA expression level of the host cell.

14. The transformed strain as claimed in claim 13, wherein the third vector is pRARE.

15. The transformed strain as claimed in claim 14, wherein the second chaperone nucleotide sequence comprises: a nucleotide sequence having at least 70% sequence identity to SEQ ID NO.: 3 and same activity with the lipase secretion chaperone protein, a nucleotide sequence having at least 70% sequence identity to SEQ ID NO.: 5 and same activity with the lipase secretion chaperone protein, a nucleotide sequence having at least 70% sequence identity to SEQ ID NO.: 6 and same activity with the lipase secretion chaperone protein, a nucleotide sequence having at least 70% sequence identity to SEQ ID NO.: 7 and same activity with the lipase secretion chaperone protein, a nucleotide sequence having at least 70% sequence identity to SEQ ID NO.: 8 and same activity with the lipase secretion chaperone protein.

16. A method of preparing PETase, comprising:
establishing a transformed strain as claimed in claim 1; and
expressing the transformed strain for obtaining the PETase.

17. A method for degrading plastic, using the transformed strain as claimed in claim 1 for degrading a plastic, wherein the plastic has PET.

18. The method as claimed in claim 17, further comprising:
an offering step: allowing the transformed strain to contact with a matrix, and inducing expression of the transformed strain so as to produce a PETase inside the transformed strain.

19. The method as claimed in claim 18, wherein the matrix comprises: solid media or liquid media.

20. The method as claimed in claim 18, further comprising:
a releasing step: disrupting the transformed strain so as to release the PETase from the transformed strain and allow the PETase to contact with the matrix, and defining a degrading composition comprising the PETase and the matrix; and
a degrading step: allowing the degrading composition to contact with the plastic.
